(19) United States
(12) Patent Application Publication
(10) Pub. No.: US 2025/0331806 A1
(43) Pub. Date: Oct. 30, 2025

(54) ULTRASOUND SYSTEM AND METHOD FOR SELECTIVE DIAGNOSIS AND TREATMENT OF PATHOGENIC CELLS

(71) Applicant: ENDOCURE LTD., Haifa (IL)

(72) Inventors: Hadas Hila Ziso, Kiryat Tivon (IL); Moshe Shoham, Hoshaya (IL)

(21) Appl. No.: 19/258,968

(22) Filed: Jul. 3, 2025

Related U.S. Application Data
(63) Continuation of application No. PCT/IB2024/050134, filed on Jan. 7, 2024.
(60) Provisional application No. 63/437,682, filed on Jan. 7, 2023.

(51) Int. Cl. A61B 8/08; A61B 8/00

(52) U.S. Cl. CPC A61B 8/085; A61B 8/40; A61B 8/4218; A61B 8/4281; A61B 8/429; A61B 8/481; A61B 8/5223; A61B 8/5246; A61B 8/54

(57) ABSTRACT

A robotic system for identifying abnormal tissue within a subject's body is provided. The robotic system includes one or more robotic arms and an ultrasound probe supporting portion that is configured to hold an ultrasound probe. The robotic system is configured to maintain the ultrasound probe at a constant orientation in space as the robotic system moves the ultrasound probe. A computer processor drives the robotic system to acquire ultrasound images of the subject's pelvis and/or abdomen while moving the ultrasound probe relative to the subject's pelvis and/or abdomen, and while maintaining the ultrasound probe in a hovering position over skin of the subject's pelvis and/or abdomen. The computer processor identifies abnormal tissue based upon the ultrasound images. Other applications are also described.

### CROSS-REFERENCES TO RELATED APPLICATIONS

The present application is a bypass continuation of International Application No. PCT/IB2024/050134 to Ziso et al. (published as WO 24/147118), filed Jan. 7, 2024, entitled “Ultrasound system and methods for selective diagnosis and treatment of pathogenic cells,” which claims priority from U.S. Provisional Patent Application No. 63/437,682 to Ziso, filed Jan. 7, 2023, entitled “System and methods for selective diagnosis and treatment of pathogenic cells by ultrasound targeted contrast agents.” The aforementioned applications are incorporated herein by reference.

### FIELD OF EMBODIMENTS OF THE INVENTION

Some applications of the present invention generally relate to medical apparatus and methods. Specifically, some applications of the present invention relate to apparatus and methods for robotic ultrasound systems for diagnosis and treatment of pathogenic cells, for example, in gynecological disorders such as endometriosis.

### BACKGROUND

Endometriosis is a progressive, chronic inflammatory disease, affecting about 200 million women of reproductive age worldwide. Endometriosis is caused by lesions of endometrium-like tissue that grow outside the uterus. These lesions affect surrounding tissue, with common sites of endometriosis being the female pelvis, the ovaries, and fallopian tubes. In some cases, endometriosis lesions affect, and sometimes block, the gastrointestinal tract, the abdomen and the urinary tract, including the kidneys and bladder.

Endometriosis lesions cause inflammation that in turn leads to fibrosis and adhesions. If left untreated, these lesions are active and the disease progresses, causing debilitating pain that profoundly affects a woman's quality of life. Additionally, endometriosis is associated with female infertility with up to 30% to 50% of women with endometriosis suffering from infertility. Endometriosis can cause fertility in several ways, such as distorted anatomy of the pelvis, adhesions, scarred fallopian tubes, and/or inflammation of the pelvic structures.

Diagnosis, as well as obtaining accurate information regarding the state of the disease, is challenging. This is partly due to endometriosis having a wide range and variability of symptoms. This is also due to a correlation between the size and/or location of the lesions and pain symptoms or infertility in endometriosis being poorly understood. Additionally, imaging has limited utility in the diagnosis of endometriosis, with the majority of the inflammation-causing lesions being invisible to current imaging modalities.

Ultrasound is typically inexpensive and readily available; however, it is typically user-dependent requiring an experienced sonographer, and even then, the data obtained is insufficient lacking adequate resolution to identify lesions, typically detecting only large lesions with a diameter of more than 5 mm. The detected large lesions are generally not indicative of the severeness and stage of the disease, thereby limiting ultrasound as an effective endometriosis detection and follow-up tool.

MRI imaging is generally more accurate than other available imaging techniques but considerably more expensive with limited availability. Even when using MRI, there are still gaps in information concerning borders and depth of infiltration of the endometriosis lesions. Additionally, detection is generally limited large lesions with a diameter of more than 5 mm.

Saliva and blood tests for biomarkers of endometriosis are entering the market, however while able to either confirm or rule out endometriosis, these tests do not provide information regarding the type, location or staging of the lesions. Thus, such biomarker testing provides only an initial step of screening with minimal effect on disease treatment and management.

Therefore, due to the limitations of imaging modalities in diagnosis of endometriosis, surgical methods, such as visual inspection by laparoscopy, preferably with confirmation of a biopsy, remain the prevailing option for a definitive diagnosis of endometriosis.

The difficulties and challenges in fully detecting endometriosis lesions badly affects the cycle of disease management, including late diagnosis of the endometriosis and impaired surgical pre-planning leading to long and complicated surgical procedures. Additionally, difficulties and challenges in fully detecting endometriosis lesions lead to unnecessary medical tests, and drug consumption, e.g., opioids.

There is therefore an ongoing need for providing non-invasive systems and techniques for accurate diagnosis and treatment of endometriosis to enable proper disease management including treatment and follow up.

### SUMMARY

In accordance with some applications of the present invention, systems and methods are provided for performing non-invasive diagnosis, and optionally treatment, of pathogenic and abnormal cells, for example, benign and/or malignant tumors. More specifically, the systems and methods provided herein are particularly configured for identification and treatment of ectopic cell lesions, such as endometriosis. For some applications, the systems provided herein are configured for high-resolution differential imaging of endometriosis lesions, and optionally, are also configured for targeted treatment of the endometriosis lesions.

The systems and methods provided in accordance with some applications of the present invention are based on a combination of robot-assisted ultrasound imaging and contrast-enhanced ultrasound imaging. Accordingly, applications of the present invention include (a) targeted and/or non-targeted ultrasound contrast agents that are administered to a subject during various stages of ultrasound scanning in order to ultimately create images in which the endometriosis lesions are enhanced within the images, and (b) robotic systems that are configured for use with ultrasound probes, and are configured to control and determine the location and orientation of the ultrasound probe as an imaging transducer in the probe performs scanning of a subject. In such a manner, the ultrasound scans are able to be processed into a series of high-resolution contrast-enhanced, three-dimensional images in which the endometriosis lesions are identifiable.

Accordingly, for some applications, an apparatus for identifying endometriosis lesions within the body of a subject, is provided. The apparatus comprises a robotic system that is configured for use with one or more ultrasound probes to create images of the endometriosis lesions for identification of the lesions. Imaging of the endometriosis lesions by the robotic system is typically enhanced by ultrasound contrast agents that are administered to the subject, and that accumulate within the lesions. The robotic system typically includes one or more robotic arms and an ultrasound probe supporting portion (typically located at a distal end of the robotic arm) that is configured to hold the ultrasound probe. The robotic system additionally includes at least one computer processor.

The computer processor is configured, prior to the contrast agent having been administered to the subject, to drive the robotic system to acquire a first set of ultrasound images of the subject's pelvis and/or abdomen while moving along a first predefined path relative to a pelvis and/or abdomen of the subject, such that the location and orientation of the ultrasound probe relative to the subject's pelvis and/or abdomen at the acquisition of each of the ultrasound images belonging to the first set of ultrasound images is known.

Subsequent to the contrast agent having been administered to the subject, the computer processor is configured to drive the robotic system to acquire a second set of ultrasound images of the subject's pelvis and/or abdomen while moving along a second predefined path relative to the subject's pelvis and/or abdomen, such that the location and orientation of the ultrasound probe relative to the subject's pelvis and/or abdomen at the acquisition of each of the ultrasound images belonging to the second set of ultrasound images is known.

The computer processor is then configured to process the acquired image data to create images in which the endometriosis lesions are identified. For some applications, data analysis and processing performed by the computer processor includes subtracting images that were acquired from each location and orientation relative to the subject's pelvis and/or abdomen within the first set of ultrasound images from the images that were acquired from the same location and orientation relative to the subject's pelvis and/or abdomen within the second set of ultrasound images, such as to generate a set of subtraction images, each of the subtraction images corresponding to a given location and orientation of the ultrasound probe relative to the subject's pelvis and/or abdomen. In such a manner, the endometriosis lesions are identifiable within the subtraction images.

Alternatively, or additionally, the data analysis and processing performed by the computer processor includes analyzing the first and second sets of ultrasound images using artificial-intelligence algorithms, e.g., for segmentation of internal organs to identify endometriosis lesions and then segmentation of the contrast-enhanced endometriosis lesions and facilitating their identification. For some applications, the computer processor runs an algorithm that has been pre-trained to identify endometriosis lesions. For example, the computer processor may run an algorithm that has pre-trained using machine-learning techniques, for example, a guided machine-learning algorithm, such as a convolutional neural network algorithm, using images of subjects' pelvises and/or abdomens acquired before and after the administration of contrast agent. For some applications, based on the pre-training, the computer processor is configured to identify endometriosis lesions based only on ultrasound images that are acquired in the absence of a contrast agent.

For some applications, a robotic system is provided comprising a tray defining internal channels, and the ultrasound probe is driven by the computer processor to perform ultrasound scanning while moving along the internal channels in the tray. Typically, for some such applications, the tray is placed on a pelvis and/or abdomen of the subject and the computer processor drives the robotic system to acquire ultrasound images of the subject's pelvis and/or abdomen while moving along the internal channels defined by the tray, such that the location and orientation of the ultrasound probe at the acquisition of each of the ultrasound images relative to the subject's pelvis and/or abdomen is known. Subsequently, the ultrasound images are processed by the computer processor for identifying endometriosis lesions based upon the ultrasound images.

For some applications, the robotic systems described herein are configured to maintain the ultrasound probe at a constant orientation in space as the robotic system moves the ultrasound probe (e.g., via the tray or by providing a robotic system having arms that include a double-parallelogram structure). For some applications, the robotic systems described herein are configured to maintain the orientation of the transducer of the ultrasound probe such that it is substantially parallel to the tangent to the center of the subject's abdomen and/or pelvis (or other scanned body area) as the robotic system moves the ultrasound probe (e.g., via the tray or by providing a robotic system having arms that include a double-parallelogram structure).

For some applications, maintaining the ultrasound probe at a constant orientation in space over the course of the procedure facilitates the generation of high-resolution three-dimensional images. For example, this typically facilitates the combination of images acquired from respective positions with each other such as to generate a three-dimensional image, because the orientations of the images in space with respect to each other are fixed. For some applications, maintaining the transducer of the ultrasound probe substantially parallel to the tangent to the center of the subject's abdomen and/or pelvis (or other scanned body area) as the robotic system moves the ultrasound probe facilitates the generation of high-resolution three-dimensional images. For example, this typically facilitates acquiring a large amount of useful and high-resolution imaging data within each image, because the ultrasound transducer is substantially directly facing the coronal plane of the subject's body at the abdomen and/or pelvis (or other scanned body area).

For some applications, a robotic system is provided for use with first and second ultrasound probes (e.g., an external ultrasound probe configured to scan above a pelvis and/or an abdomen or a back of the subject, and an intraluminal ultrasound probe configured to be inserted into a vagina or rectum of the subject). For some such applications, a computer processor of the robotic system is configured to determine the positions and orientations of the two ultrasound probes with respect to each other, and to drive the ultrasound probes to acquire ultrasound images to generate three-dimensional ultrasonic imaging data based on a combination of pelvic/abdominally-acquired ultrasound images and the intraluminally-acquired ultrasound images to identify endometriosis lesions based upon the three-dimensional ultrasonic imaging data. Additional robotic systems are described herein.

For some applications, the robotic systems and methods described herein are configured for diagnosis, and optionally treatment of any type of ectopic tissue. Additionally, or alternatively, the robotic systems and methods described herein are configured for diagnosis, and optionally treatment of tumors, both benign and malignant, inflammation, thrombi or any other types of pathogenic cells or lesions within a subject's body.

There is therefore provided in accordance with some applications of the present invention, apparatus for identifying endometriosis lesions within a body of a subject, and for use with at least one ultrasound probe and a contrast agent configured to enhance the endometriosis lesions within ultrasound images, the apparatus including:
a robotic system including:
   one or more robotic arms; and
   an ultrasound probe supporting portion that is configured to hold the ultrasound probe; and
at least one computer processor configured:
   prior to the contrast agent having been administered to the subject, to drive the robotic system to acquire a first set of ultrasound images of the subject's pelvis and/or abdomen while moving along a first predefined path, such that the location and orientation of the ultrasound probe in space at the acquisition of each of the ultrasound images belonging to the first set of ultrasound images is known;
   subsequent to the contrast agent having been administered to the subject, to drive the robotic system to acquire a second set of ultrasound images of the subject's pelvis and/or abdomen while moving along a second predefined path, such that the location and orientation of the ultrasound probe in space at the acquisition of each of the ultrasound images belonging to the second set of ultrasound images is known;
   to subtract images that were acquired from each location and orientation in space relative to within the first set of ultrasound images from the images that were acquired from the same location and orientation in space within the second set of ultrasound images, such as to generate a set of subtraction images, each of the subtraction images corresponding to a given location and orientation of the ultrasound probe in space; and
   to identify endometriosis lesions within the subtraction images.

For some applications, the first predefined path is the same as the second predefined path.

For some applications, the robotic system is configured to maintain the ultrasound probe at a constant orientation in space as the robotic system moves the ultrasound probe along the first and second predefined paths.

For some applications, the ultrasound probe includes a transducer and the robotic system is configured to maintain the orientation of the transducer of the ultrasound probe such that it is substantially parallel to a tangent to a center of the pelvis and/or an abdomen of the subject as the robotic system moves the ultrasound probe.

For some applications, the ultrasound probe includes a transducer and the robotic system is configured to maintain the orientation of the transducer of the ultrasound probe such that the ultrasound transducer is substantially directly facing a coronal plane of the subject's body at the subject's abdomen and/or pelvis as the robotic system moves the ultrasound probe.

For some applications, the computer processor is configured to drive the ultrasound probe to apply ablative ultrasound energy to the endometriosis lesions while moving along one of the first and second predefined paths in response to identifying the endometriosis lesions.

For some applications, the apparatus includes the contrast agent.

For some applications, the contrast agent includes a contrast agent selected from the group consisting of: targeted contrast agents and non-targeted contrast agents.

For some applications, the contrast agent includes microbubbles configured to enhance the endometriosis lesions within ultrasound images, and the computer processor is configured to drive the ultrasound probe to apply targeted focused ultrasound to cause cavitation of tissue in the vicinity of the microbubbles.

For some applications, the robotic system includes a tray configured to be placed on the pelvis and/or abdomen of the subject, the tray defining internal channels which define the first and second predefined paths, and
the ultrasound probe supporting portion is configured to hold the ultrasound probe and to move the ultrasound probe along the internal channels defined by the tray; and
the at least one computer processor is configured to drive the robotic system to acquire the first and second sets of ultrasound images of the subject's pelvis and/or abdomen while moving along the internal channels defined by the tray, such that the location and orientation of the ultrasound probe at the acquisition of each of the ultrasound images relative to the subject's pelvis and/or abdomen is known.

For some applications, the robotic system is configured to maintain the ultrasound probe at a constant orientation in space as the robotic system moves the ultrasound probe along the channels defined by the tray.

For some applications, the ultrasound probe includes a transducer and the robotic system is configured to maintain an orientation of the ultrasound transducer of the ultrasound probe such that it is substantially parallel to a tangent to a center of the subject's pelvis and/or abdomen as the robotic system moves the ultrasound probe.

For some applications, the ultrasound probe includes a transducer and the robotic system is configured to maintain an orientation of the ultrasound transducer of the ultrasound probe such that the ultrasound transducer is substantially directly facing a coronal plane of the subject's body at the subject's abdomen and/or pelvis as the robotic system moves the ultrasound probe.

For some applications, the tray is configured to be attached to the subject, such that the tray moves with the subject.

For some applications, the tray is shaped to define a curved tray configured to conform to a shape of the pelvis and/or abdomen of the subject, such that when placed on the subject's abdomen and/or pelvis the tray is coupled to skin of the subject generally without gaps between the tray and the subject's skin.

For some applications, a surface of the tray that is placed on the subject's pelvis and/or abdomen is made of an ultrasound transparent material.

For some applications, the tray is shaped to define a flat base, the flat base configured to maintain the ultrasound probe at a constant orientation in space as the robotic system moves the ultrasound probe.

For some applications, the flat base of the tray is shaped to define a flat base, the flat base configured to maintain the ultrasound probe substantially parallel to a tangent to a center of the subject's pelvis and/or abdomen as the robotic system moves the ultrasound probe.

For some applications, the apparatus includes a water-filled compartment configured to be placed between the subject's abdomen and/or pelvis and the flat base of the tray.

For some applications, the one or more robotic arms include a double-parallelogram structure configured to maintain the ultrasound probe at a constant orientation in space as the robotic system moves the ultrasound probe.

For some applications, the ultrasound probe includes a transducer, and the one or more robotic arms include a double-parallelogram structure configured to maintain an orientation of the transducer of the ultrasound probe such that it is substantially parallel to a tangent to a center of the subject's abdomen and/or pelvis as the robotic system moves the ultrasound probe.

For some applications, the ultrasound probe includes a transducer, and the one or more robotic arms include a double-parallelogram structure configured to maintain an orientation of the transducer of the ultrasound probe such that the ultrasound transducer is substantially directly facing a coronal plane of the subject's body at the subject's abdomen and/or pelvis as the robotic system moves the ultrasound probe.

There is further provided in accordance with some applications of the present invention, a method for identifying endometriosis lesions within a body of a subject, and for use with at least one ultrasound probe and a contrast agent configured to enhance the endometriosis lesions within ultrasound images, the method including:
using a robotic system, holding the ultrasound probe by an ultrasound probe supporting portion coupled to one or more robotic arms of the robotic system; and
using at least one computer processor:
   prior to the contrast agent having been administered to the subject, driving the robotic system to acquire a first set of ultrasound images of the subject's pelvis and/or abdomen while moving along a first predefined path, such that the location and orientation of the ultrasound probe in space at the acquisition of each of the ultrasound images belonging to the first set of ultrasound images is known;
   subsequent to the contrast agent having been administered to the subject, driving the robotic system to acquire a second set of ultrasound images of the subject's pelvis and/or abdomen while moving along a second predefined path, such that the location and orientation of the ultrasound probe in space at the acquisition of each of the ultrasound images belonging to the second set of ultrasound images is known;
   subtract images that were acquired from each location and orientation in space within the first set of ultrasound images from the images that were acquired from the same location and orientation in space within the second set of ultrasound images, such as to generate a set of subtraction images, each of the subtraction images corresponding to a given location and orientation of the ultrasound probe in space; and
   identify endometriosis lesions within the subtraction images.

There is further provided in accordance with some applications of the present invention apparatus for identifying endometriosis lesions within a body of a subject, and for use with at least one ultrasound probe, the apparatus including:
a robotic system including:
   one or more robotic arms; and
   an ultrasound probe supporting portion that is configured to hold the ultrasound probe;
   the robotic system being configured to maintain the ultrasound probe at a constant orientation in space as the robotic system moves the ultrasound probe; and at least one computer processor configured:
   to drive the robotic system to acquire ultrasound images of the subject's pelvis and/or abdomen while moving relative to the subject's pelvis and/or abdomen, such that the location and orientation of the ultrasound probe in space at the acquisition of each of the ultrasound images is known; and
   to identify endometriosis lesions based upon the ultrasound images.

For some applications, the ultrasound probe includes a transducer and the robotic system is configured to maintain the orientation of the transducer of the ultrasound probe such that it is substantially parallel to a tangent to a center of the subject's pelvis and/or an abdomen as the robotic system moves the ultrasound probe.

For some applications, the ultrasound probe includes a transducer and the robotic system is configured to maintain the orientation of the transducer of the ultrasound probe such that the ultrasound transducer is substantially directly facing a coronal plane of the subject's body at the subject's abdomen and/or pelvis as the robotic system moves the ultrasound probe.

For some applications, the robotic system includes a tray configured to be placed on the subject's pelvis and/or abdomen, the tray defining internal channels, and
the ultrasound probe supporting portion is configured to hold the ultrasound probe and to move the ultrasound probe along the internal channels defined by the tray; and
the at least one computer processor is configured to drive the robotic system to acquire the ultrasound images while moving along the internal channels defined by the tray, such that the location and orientation of the ultrasound probe at the acquisition of each of the ultrasound images relative to the subject's pelvis and/or abdomen is known.

For some applications, the tray is configured to maintain the ultrasound probe at the constant orientation in space as the robotic system moves the ultrasound probe along the channels defined by the tray.

For some applications, the ultrasound probe includes a transducer, and the tray is configured to maintain an orientation of an ultrasound transducer of the ultrasound probe such that it is substantially parallel to a tangent to a center of the subject's pelvis and/or abdomen as the robotic system moves the ultrasound probe.

For some applications, the tray is configured to maintain an orientation of an ultrasound transducer of the ultrasound probe such that the ultrasound transducer is substantially directly facing a coronal plane of the subject's body at the subject's abdomen and/or pelvis as the robotic system moves the ultrasound probe.

For some applications, the tray is configured to be attached to the subject, such that the tray moves with the subject.

For some applications, the tray is shaped to define a curved tray configured to conform to a shape of the pelvis and/or abdomen of the subject, such that when placed on the subject's pelvis and/or abdomen the tray is coupled to skin of the subject generally without gaps between the tray and the subject's skin.

For some applications, a surface of the tray that is placed on the pelvis and/or abdomen of the subject is made of an ultrasound transparent material.

For some applications, the tray is shaped to define a flat base, the flat base configured to maintain the ultrasound probe at the constant orientation in space as the robotic system moves the ultrasound probe.

For some applications, the ultrasound probe includes a transducer, and the flat base of the tray is shaped to define a flat base, the flat base configured to maintain an orientation of the transducer of the ultrasound probe such that the transducer is substantially parallel to a tangent to a center of the subject's pelvis and/or abdomen as the robotic system moves the ultrasound probe.

For some applications, the apparatus further includes a water-filled compartment configured to be placed between the subject's pelvis and/or abdomen and the flat base of the tray.

For some applications, the flat base is configured to maintain an orientation of the transducer of the ultrasound probe such that the transducer is substantially directly facing a coronal plane of the subject's body at the subject's abdomen and/or pelvis as the robotic system moves the ultrasound probe.

For some applications, the apparatus is for use with a contrast agent configured to enhance the endometriosis lesions within ultrasound images, and the computer processor is configured to:
prior to the contrast agent having been administered to the subject, to drive the robotic system to acquire a first set of ultrasound images of the subject's pelvis and/or abdomen by moving the ultrasound probe while maintaining the probe at the constant orientation at the acquisition of each of the ultrasound images belonging to the first set of ultrasound images;
subsequent to the contrast agent having been administered to the subject, to drive the robotic system to acquire a second set of ultrasound images of the subject's pelvis and/or abdomen by moving the ultrasound probe while maintaining the probe at the constant orientation at the acquisition of each of the ultrasound images belonging to the second set of ultrasound images is known; and
identify the endometriosis lesions by analyzing the first and second sets of ultrasound images.

For some applications, the computer processor is configured to subtract images that were acquired within the first set of ultrasound images from the images that were acquired within the second set of ultrasound images, such as to generate a set of subtraction images, each of the subtraction images corresponding to a given location and orientation of the ultrasound probe in space.

For some applications, the contrast agent is selected from the group consisting of: targeted contrast agents and non-targeted contrast agents.

For some applications, the apparatus further includes the contrast agent.

For some applications, the contrast agent includes microbubbles configured to enhance the endometriosis lesions within ultrasound images, and the computer processor is configured to drive the ultrasound probe to apply targeted focused ultrasound to cause cavitation of tissue in the vicinity of the microbubbles.

For some applications, the one or more robotic arms include a double-parallelogram structure configured to maintain the ultrasound probe at the constant orientation in space as the robotic system moves the ultrasound probe.

For some applications, the ultrasound probe includes a transducer, and the one or more robotic arms include a double-parallelogram structure configured to maintain an orientation of the transducer of the ultrasound probe such that it is substantially parallel to a tangent to a center of the subject's pelvis and/or abdomen as the robotic system moves the ultrasound probe.

For some applications, the ultrasound probe includes a transducer, and the one or more robotic arms include a double-parallelogram structure configured to maintain an orientation of the transducer of the ultrasound probe such that the transducer is substantially directly facing a coronal plane of the subject's body at the subject's abdomen and/or pelvis as the robotic system moves the ultrasound probe.

For some applications, the computer processor is further configured to drive the ultrasound probe to apply ablative ultrasound energy to the endometriosis lesions while moving the ultrasound probe relative to the subject's pelvis and/or abdomen, at a position and orientation of the ultrasound probe that is the same as the location and orientation of the ultrasound probe at acquisitions of the ultrasound images, in response to identifying the endometriosis lesions.

There is further provided in accordance with some applications of the present invention, apparatus for identifying endometriosis lesions within a body of a subject, and for use with at least one ultrasound probe, the apparatus including:
a robotic system including:
   a tray configured to be placed on a pelvis and/or an abdomen of the subject, the tray defining internal channels; and
   an ultrasound probe supporting portion that is configured to hold the ultrasound probe and to move along the internal channels defined by the tray; and at least one computer processor configured:
   to drive the robotic system to acquire ultrasound images of the subject's pelvis and/or abdomen while moving along the internal channels defined by the tray, such that the location and orientation of the ultrasound probe at the acquisition of each of the ultrasound images relative to the subject's pelvis and/or abdomen is known; and
   to identify endometriosis lesions based upon the ultrasound images.

For some applications, the tray is configured to maintain the ultrasound probe at a constant orientation in space as the robotic system moves the ultrasound probe along the channels defined by the tray.

For some applications, the ultrasound probe includes a transducer, and the tray is configured to maintain an orientation of an ultrasound transducer of the ultrasound probe such that it is substantially parallel to a tangent to a center of the pelvis and/or abdomen of the subject as the robotic system moves the ultrasound probe.

For some applications, the tray is configured to be attached to the subject, such that the tray moves with the subject.

For some applications, the tray is shaped to define a curved tray configured to conform to a shape of the pelvis and/or abdomen of the subject, such that when placed on the pelvis and/or abdomen the tray is coupled to the skin of the subject generally without gaps between the tray and the subject.

For some applications, a surface of the tray that is placed on the pelvis and/or abdomen of the subject is made of an ultrasound transparent material.

For some applications, the tray is shaped to define a flat base, the flat base configured to maintain the ultrasound probe at a constant orientation in space as the robotic system moves the ultrasound probe.

For some applications, the computer processor is further configured to drive the ultrasound probe to apply ablative ultrasound energy to the endometriosis lesions while moving along the internal channels defined by the tray in response to identifying the endometriosis lesions.

For some applications, the internal channels are separated from each other by at least one wall defining a groove, and the ultrasound probe is configured to move along the internal channels by one or more protrusions on the ultrasound probe sliding along the groove in the wall.

For some applications, the tray is shaped to define a flat base configured to maintain the ultrasound probe substantially parallel to a tangent to a center of the pelvis and/or abdomen of the subject as the robotic system moves the ultrasound probe.

For some applications, the apparatus further includes a water-filled compartment configured to be placed between the pelvis and/or abdomen of the subject and the flat base of the tray.

For some applications, the apparatus is for use with a contrast agent configured to enhance the endometriosis lesions within ultrasound images, the contrast agent selected from the group consisting of: targeted contrast agents and non-targeted contrast agents.

For some applications, the apparatus further includes the contrast agent.

For some applications, the contrast agent includes microbubbles configured to enhance the endometriosis lesions within ultrasound images, and the computer processor is configured to drive the ultrasound probe to apply targeted focused ultrasound to cause cavitation of tissue in the vicinity of the microbubbles.

There is further provided in accordance with some applications of the present invention, apparatus for identifying endometriosis lesions within a body of a subject, and for use with first and second ultrasound probes, the apparatus including:
a robotic system including:
   an abdominal portion including one or more abdominal robotic arms and an abdominal ultrasound probe supporting portion that is configured to hold the first ultrasound probe above an abdomen and/or a pelvis of the subject; and
   an intraluminal portion including one or more intraluminal robotic arms and an intraluminal ultrasound probe supporting portion, the intraluminal portion being configured to insert the second ultrasound probe into a lumen of the subject selected from the group consisting of: a rectum and a vagina.
at least one computer processor configured to:
   determine the positions and orientations of the first and second ultrasound probes with respect to each other;
   drive the first ultrasound probe to acquire abdominally-acquired ultrasound images of the subject's pelvis and/or abdomen while driving the abdominal portion of the robotic system to move the first ultrasound probe relative to the subject's pelvis and/or abdomen; and
   drive the second ultrasound probe to acquire intraluminally-acquired ultrasound images of the subject's pelvis and/or abdomen while driving the intraluminal portion of the robotic system to move the second ultrasound probe relative to the subject's pelvis and/or abdomen;
   generate three-dimensional ultrasonic imaging data based on a combination of the abdominally-acquired ultrasound images and the intraluminally-acquired ultrasound images; and
   identify endometriosis lesions based upon the three-dimensional ultrasonic imaging data.

For some applications, the robotic system is configured to maintain the first ultrasound probe at a constant orientation in space as the robotic system drives the abdominal portion of the robotic system to move the first ultrasound probe relative to the subject's pelvis and/or abdomen; and
the computer processor is configured to determine the position and orientation of the first and second ultrasound probes relative to each other by using the constant orientation in space of the first ultrasound probe.

For some applications, the robotic system is configured to maintain the second ultrasound probe at a constant orientation in space as the robotic system drives the intraluminal portion of the robotic system to move the second ultrasound probe relative to the subject's pelvis and/or abdomen; and
the computer processor is configured to determine the position and orientation of the first and second ultrasound probes relative to each other by using the constant orientation in space of the second ultrasound probe.

For some applications, the robotic system is configured to maintain the first ultrasound probe at a constant orientation in space as the robotic system drives the abdominal portion of the robotic system to move the first ultrasound probe relative to the subject's pelvis and/or abdomen;
the robotic system is configured to maintain the second ultrasound probe at a constant orientation in space as the robotic system drives the intraluminal portion of the robotic system to move the second ultrasound probe relative to the subject's pelvis and/or abdomen; and
the computer processor is configured to determine the position and orientation of each of the first and second ultrasound probes relative to each other by using the constant orientation in space of the first and second ultrasound probes.

For some applications, the computer processor is further configured to drive the first and or the second ultrasound probes to apply ablative ultrasound energy to the endometriosis lesions while driving the abdominal portion and/or the intraluminal portion of the robotic system to move the first and/or the ultrasound probe relative to the subject's pelvis and/or abdomen.

For some applications, the apparatus is for use with a contrast agent configured to enhance the endometriosis lesions within ultrasound images, the contrast agent selected from the group consisting of: targeted contrast agents and non-targeted contrast agents.

For some applications, the robotic system includes a base and the abdominal robotic arms and the intraluminal robotic arms are both mounted to the base, and the computer processor is configured to determine the position and orientation of each of the first and second ultrasound probes relative to each other by determining the position and orientation of each of the first and second ultrasound probes relative to the base.

There is further provided, in accordance with some embodiments of the present invention, apparatus for identifying endometriosis lesions within a body of a subject, and for use with at least one ultrasound probe, the apparatus including:
a robotic system including:
   one or more robotic arms; and
   an ultrasound probe supporting portion that is configured to hold the ultrasound probe; and
at least one computer processor configured:
   to drive the robotic system to acquire ultrasound images of the subject's pelvis and/or abdomen while moving the ultrasound probe relative to the subject's pelvis and/or abdomen, and while maintaining the ultrasound probe in a hovering position over skin of the subject's pelvis and/or abdomen; and
   to identify endometriosis lesions based upon the ultrasound images.

For some applications, the computer processor is configured to avoid movement and deformation of the skin and underlying body structures undergoing scanning by the ultrasound probe by maintaining the ultrasound probe in the hovering position over skin of the subject's pelvis and/or abdomen.

For some applications, the apparatus further includes a sensor configured to monitor a distance between the ultrasound probe and the skin, the computer processor is configured to maintain the ultrasound probe in a hovering position over skin of the subject's pelvis and/or abdomen based upon the distance between the ultrasound probe and the skin as monitored by the sensor.

For some applications, the ultrasound probe includes a transducer and the robotic system is configured to maintain an orientation of the transducer of the ultrasound probe such that it is substantially parallel to a tangent to a center of the subject's pelvis and/or an abdomen as the robotic system moves the ultrasound probe.

For some applications, the ultrasound probe includes a transducer and the robotic system is configured to maintain an orientation of the transducer of the ultrasound probe such that the ultrasound transducer is substantially directly facing a coronal plane of the subject's body at the subject's abdomen and/or pelvis as the robotic system moves the ultrasound probe.

For some applications, the robotic system is configured to maintain the ultrasound probe at a constant orientation in space as the robotic system moves the ultrasound probe.

For some applications:
the robotic system includes a tray configured to be placed on the subject's pelvis and/or abdomen, the tray defining internal channels, and
the ultrasound probe supporting portion is configured to hold the ultrasound probe and to move the ultrasound probe along the internal channels defined by the tray; and
the at least one computer processor is configured to drive the robotic system to acquire the ultrasound images while moving along the internal channels defined by the tray, such that the location and orientation of the ultrasound probe at the acquisition of each of the ultrasound images relative to the subject's pelvis and/or abdomen is known.

For some applications, the tray is configured to maintain the ultrasound probe at a constant orientation in space as the robotic system moves the ultrasound probe along the channels defined by the tray.

For some applications, the ultrasound probe includes a transducer, and the tray is configured to maintain an orientation of an ultrasound transducer of the ultrasound probe such that it is substantially parallel to a tangent to a center of the subject's pelvis and/or abdomen as the robotic system moves the ultrasound probe.

For some applications, the tray is configured to maintain an orientation of an ultrasound transducer of the ultrasound probe such that the ultrasound transducer is substantially directly facing a coronal plane of the subject's body at the subject's abdomen and/or pelvis as the robotic system moves the ultrasound probe.

For some applications, the tray is configured to be attached to the subject, such that the tray moves with the subject.

For some applications, the tray is shaped to define a curved tray configured to conform to a shape of the pelvis and/or abdomen of the subject, such that when placed on the subject's pelvis and/or abdomen the tray is coupled to skin of the subject generally without gaps between the tray and the subject's skin.

For some applications, a surface of the tray that is placed on the pelvis and/or abdomen of the subject is made of an ultrasound transparent material.

For some applications, the tray is shaped to define a flat base, the flat base configured to maintain the ultrasound probe at the constant orientation in space as the robotic system moves the ultrasound probe.

For some applications, the ultrasound probe includes a transducer, and the flat base of the tray is shaped to define a flat base, the flat base configured to maintain an orientation of the transducer of the ultrasound probe such that the transducer is substantially parallel to a tangent to a center of the subject's pelvis and/or abdomen as the robotic system moves the ultrasound probe.

For some applications, the apparatus further includes a water-filled compartment configured to be placed between the subject's pelvis and/or abdomen and the flat base of the tray.

For some applications, the flat base configured to maintain an orientation of the transducer of the ultrasound probe such that the transducer is substantially directly facing a coronal plane of the subject's body at the subject's abdomen and/or pelvis as the robotic system moves the ultrasound probe.

For some applications, the apparatus is for use with a contrast agent configured to enhance the endometriosis lesions within ultrasound images, and the computer processor is configured to:
prior to the contrast agent having been administered to the subject, to drive the robotic system to acquire a first set of ultrasound images of the subject's pelvis and/or abdomen while moving the ultrasound probe;
subsequent to the contrast agent having been administered to the subject, to drive the robotic system to acquire a second set of ultrasound images of the subject's pelvis and/or abdomen while moving the ultrasound probe; and
identify the endometriosis lesions by analyzing the first and second sets of ultrasound images.

For some applications, the contrast agent is selected from the group consisting of: targeted contrast agents and non-targeted contrast agents.

For some applications, the apparatus further includes the contrast agent.

For some applications, the contrast agent includes microbubbles configured to enhance the endometriosis lesions within ultrasound images, and the computer processor is configured to drive the ultrasound probe to apply targeted focused ultrasound to cause cavitation of tissue in the vicinity of the microbubbles.

For some applications, by avoiding movement and deformation of the skin and underlying body structures undergoing scanning by the probe, the computer processor is configured to minimize changes in position and shape of the skin and underlying body structures between the acquisitions of the first and second sets of ultrasound images of the subject's pelvis and/or abdomen.

For some applications, the computer processor is configured to subtract images that were acquired within the first set of ultrasound images from the images that were acquired within the second set of ultrasound images, such as to generate a set of subtraction images, each of the subtraction images corresponding to a given location and orientation of the ultrasound probe in space.

For some applications, by avoiding movement and deformation of the skin and underlying body structures undergoing scanning by the probe, the computer processor is configured to minimize changes in position and shape of the skin and underlying body structures between the acquisitions of the first and second sets of ultrasound images of the subject's pelvis and/or abdomen, to thereby enhance accuracy of the subtraction images.

For some applications, the one or more robotic arms include a double-parallelogram structure configured to maintain the ultrasound probe at a constant orientation in space as the robotic system moves the ultrasound probe.

For some applications, the ultrasound probe includes a transducer, and the one or more robotic arms include a double-parallelogram structure configured to maintain an orientation of the transducer of the ultrasound probe such that it is substantially parallel to a tangent to a center of the subject's pelvis and/or abdomen as the robotic system moves the ultrasound probe.

For some applications, the ultrasound probe includes a transducer, and the one or more robotic arms include a double-parallelogram structure configured to maintain an orientation of the transducer of the ultrasound probe such that the transducer is substantially directly facing a coronal plane of the subject's body at the subject's abdomen and/or pelvis as the robotic system moves the ultrasound probe.

For some applications, the computer processor is further configured to drive the ultrasound probe to apply ablative ultrasound energy to the endometriosis lesions while moving the ultrasound probe relative to the subject's pelvis and/or abdomen, at a position and orientation of the ultrasound probe that is the same as the location and orientation of the ultrasound probe at acquisitions of the ultrasound images, in response to identifying the endometriosis lesions.

There is further provided in accordance with some applications of the present invention, apparatus for identifying endometriosis lesions within a body of a subject, and for use with at least one ultrasound probe and a contrast agent configured to enhance the endometriosis lesions within ultrasound images, the apparatus including:
a robotic system including:
   one or more robotic arms; and
   an ultrasound probe supporting portion that is configured to hold the ultrasound probe; and
at least one computer processor configured:
   prior to the contrast agent having been administered to the subject, to drive the robotic system to acquire a first set of ultrasound images of the subject's pelvis and/or abdomen while moving along a first predefined path, such that the location and orientation of the ultrasound probe in space at the acquisition of each of the ultrasound images belonging to the first set of ultrasound images is known;
   subsequent to the contrast agent having been administered to the subject, to drive the robotic system to acquire a second set of ultrasound images of the subject's pelvis and/or abdomen while moving along a second predefined path, such that the location and orientation of the ultrasound probe in space at the acquisition of each of the ultrasound images belonging to the second set of ultrasound images is known; and
   to identify the endometriosis lesions by analyzing the first and second sets of ultrasound images.

The present invention will be more fully understood from the following detailed description of embodiments thereof, taken together with the drawings, in which:

### BRIEF DESCRIPTION OF THE DRAWINGS

FIG. 1A is a block diagram showing components of a robotic system that is configured for use for identifying endometriosis lesions within a body of a subject, in accordance with some applications of the present invention;

FIG. 1B is a flowchart showing steps of methods that are performed, in accordance with some applications of the present invention;

FIG. 2 is a schematic illustration of an apparatus comprising a robotic system configured for use for identifying endometriosis lesions within a body of a subject, in accordance with some applications of the present invention;

FIGS. 3A and 3B are schematic illustrations of components of a robotic system configured for use for identifying endometriosis lesions within a body of a subject, in accordance with some applications of the present invention;

FIGS. 4A and 4B are schematic illustrations of components of a robotic system configured for use for identifying endometriosis lesions within a body of a subject, in accordance with some applications of the present invention;

FIGS. 5A and 5B are schematic illustrations of components of a robotic system configured for use for identifying endometriosis lesions within a body of a subject, in accordance with some applications of the present invention;

FIGS. 6A and 6B are schematic illustrations of components of a robotic system configured for use for identifying endometriosis lesions within a body of a subject, in accordance with some applications of the present invention;

FIG. 7 is a schematic illustration of an apparatus comprising a robotic system for identifying endometriosis lesions within a body of a subject configured for use with a water-filled compartment, in accordance with some applications of the present invention;

FIG. 8A is a schematic illustration of an apparatus comprising a robotic system for identifying endometriosis lesions within a body of a subject, and configured for use with an abdominal ultrasound probe and an intraluminal ultrasound probe, in accordance with some applications of the present invention;

FIG. 8B is a schematic illustration of the apparatus of FIG. 8A being used to scan a subject in accordance with some applications of the present invention;

FIGS. 9A and 9B are schematic illustrations of an apparatus configured for use with an ultrasound probe and comprising a robotic system for identifying endometriosis lesions within a body of a subject, and structured to maintain the ultrasound probe at a constat orientation in space as the robotic system moves the ultrasound probe, in accordance with some applications of the present invention; and

FIG. 10 is a table showing experimental results of experiments performed by the inventors in accordance with some applications of the present invention and using the apparatus, robotic systems and techniques described herein.

### DETAILED DESCRIPTION OF EMBODIMENTS

In accordance with some applications of the present invention, systems and methods are provided for performing non-invasive diagnosis and optionally treatment of pathogenic and abnormal cells, for example, inflamed tissue, thrombus, benign and/or malignant tumors. More specifically, the systems and methods provided herein are configured for identification and treatment of ectopic cell lesions, including, but not limited to, endometriosis. For some applications, the system comprises an ultrasound system configured for high resolution selective imaging of endometriosis lesions, and optionally also configured for targeted treatment of the endometriosis lesions.

Typically, the ultrasound system comprises a robotic ultrasound system. In an imaging mode the ultrasound system is configured to detect the presence of endometriosis lesions, and in a therapeutic mode the ultrasound system is configured to apply treatment energy at the detected lesions for performing a therapeutic procedure, such as ablation. For some applications, the ultrasound system comprises an ultrasound probe configured to alternate between an imaging mode and a treatment mode. Alternatively, the ultrasound system comprises separate probes for imaging and treatment. Typically, the ultrasound probes are operated and controlled by a robotic system as will be described in further detail.

In accordance with some applications of the present invention, the robotic ultrasound system is configured for comprehensive managing of endometriosis, including: (a) detection and early differential diagnosis of endometriosis; (b) personalized treatment planning based on the imaging; (c) monitoring the effectiveness of the treatment by regular follow-up scans of the endometriosis lesions; and (d) optionally, the system is also configured to apply treatment for treating the lesions.

### Imaging and Diagnosis

The robotic imaging ultrasound system provided in accordance with some applications of the present invention, is configured to detect the presence of endometriosis lesions, to obtain a definitive diagnosis of endometriosis.

Additionally, the system can be used for pre-operative imaging to detect, map and/or confirm the presence, size and location of lesion tissue, before the execution of any treatment procedures such as ablative procedures, or other surgical procedures. Pre-operative imaging typically contributes to performing safer and more effective treatment procedures, by typically reducing the duration of the surgical procedures and by increasing selectivity of the procedure thereby avoiding damage of healthy tissue adjacent to the lesions.

As described herein above, for some applications, the imaging system combines robotic-assisted ultrasound imaging with the use of ultrasound contrast agents. The robot-assisted ultrasound system is configured to detect endometriosis lesions by creating an image series (for example, in a manner similar to techniques used in CT and MRI imaging) by processing the image data to create high-resolution three-dimensional reconstruction of the contrast-enhanced ultrasound images.

More specifically, for some applications, the ultrasound imaging is performed prior to, and again subsequently to, injection of targeted and/or non-targeted ultrasound contrast agents that accumulate within the endometriosis lesion(s, such that identification of the lesions is enhanced by the contrast agents. Use of contrast agents typically facilitates detection of superficial lesions that are generally not detectable by using standard available imaging techniques. Additionally, use of the contrast agents improves detection and assessment of deep lesions. For some applications, use of the contrast agents alone does not enable optimal detection of the endometriosis lesions using standard available imaging techniques. Thus, in accordance with some applications of the present invention, the use of ultrasound contrast agents is combined with a robotic system, e.g., comprising a robotic arm, that is coupled to an existing ultrasound imaging transducer/probe for scanning the area of concern (e.g., scanning the pelvis and/or abdomen of a subject using an external ultrasound transducer) before and after administration of the contrast agent.

Typically, the robotic ultrasound system includes one or more computer processors that process the scanned data that is streamed to the computer processor. The data is processed by the computer processor into a series of three-dimensional images (e.g., three-dimensional DICOM images), similar to images obtained by MRI or CT scans. Then, enhanced lesion identification is performed by further processing. For some applications, processing of the scanned data is based on subtraction of the data acquired before and after administration of the contrast agents to eliminate the background of healthy tissue.

Alternatively, or additionally, the data analysis and processing performed by the computer processor includes analyzing the first and second sets of ultrasound images using artificial-intelligence algorithms, e.g., for segmentation of internal organs for detection of the lesions within the images, and segmentation of the contrast-enhanced endometriosis lesions and facilitating their identification. For some applications, the computer processor runs an algorithm that has been pre-trained to identify endometriosis lesions. For example, the computer processor may run an algorithm that has pre-trained using machine-learning techniques, for example, a guided machine-learning algorithm, such as a convolutional neural network algorithm, using images of subjects' pelvises and/or abdomens acquired before and after the administration of contrast agent. For some applications, based on the pre-training, the computer processor is configured to identify endometriosis lesions based only on ultrasound images that are acquired in the absence of contrast agent (such that use of contrast agents is not required, and the robotic systems described herein are configured to detect endometriosis and other lesions without the use of contrast agents).

Identification of the lesions include, for example, determining accurate volume measurements of the lesions and complete mapping of the lesions (such that a health professional can determine the stage of the disease, and an appropriate treatment plan).

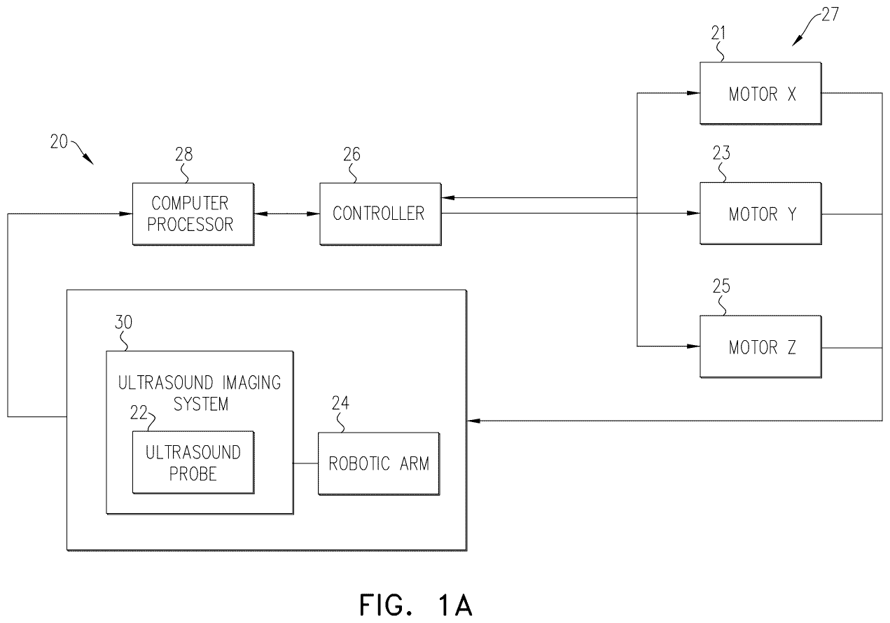

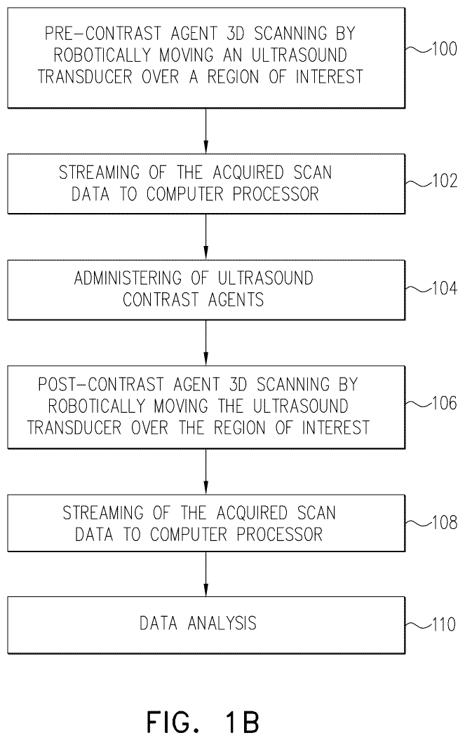

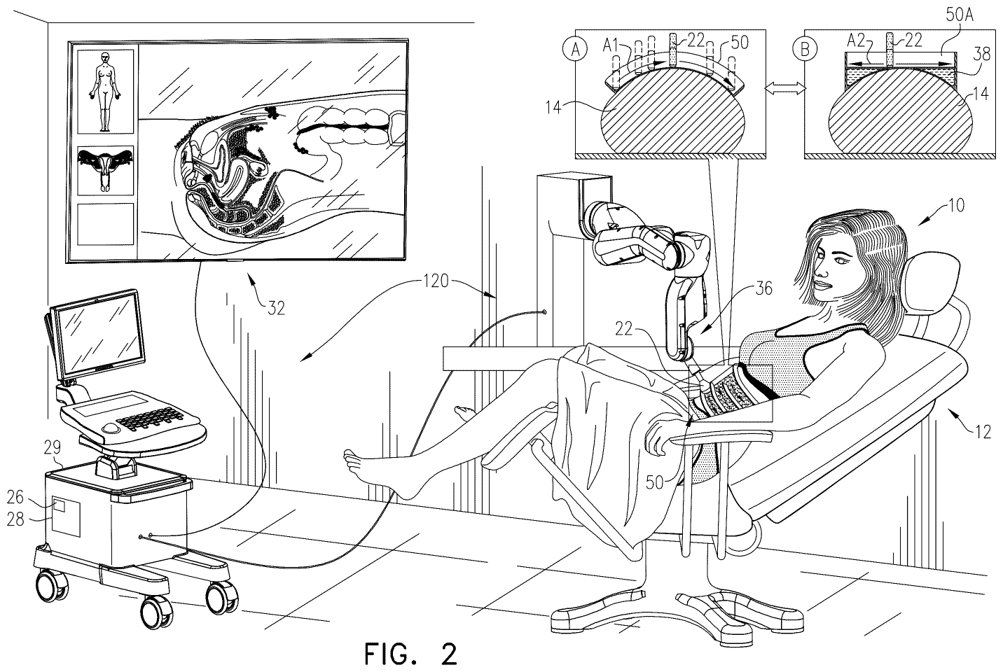

Reference is now made to FIG. 1A, which is a block diagram showing components of an ultrasound robotic system 20 that is configured for use in an imaging procedure for imaging of endometriosis lesions in subject 10 (subject 10 is shown in FIG. 2), in accordance with some applications of the present invention.

Typically, when used for imaging, robotic system 20 is configured for use with an ultrasound probe 22 of ultrasound imaging system 30 configured to scan the patient. Robotic system 20 comprises one or more robotic arms 24 that hold ultrasound probe 22 (either directly or via an ultrasound probe supporting portion) and is configured to be moved by motor system 27 to move ultrasound probe 22 with respect to the subject. For some applications, motor system 27 that moves robotic arm 24 comprises XYZ motors 21, 23, 25, respectively, that provide three degrees-of-freedom. For some applications, the robotic system additional includes motors that are configured to provide angular rotations, e.g., motors 152 (shown in FIGS. 4A-4B, for example), e.g., roll, pitch and/or yaw angular rotations. Typically, these motors are configured to provide angular rotations that are such as to maintain the ultrasound probe at a constant orientation in space as the robotic system moves the ultrasound probe, as described in further detail hereinbelow. For some such applications, the motors are configured to provide six degrees-of-freedom. Motors 21, 23 and 25, and any additional motors, are collectively referred to as motor system 27. Typically, there is an encoder, e.g., a rotary encoder, or a different sensor, associated with the degree-of-freedom controlled by each of the motors, such that the position of the robotic arm can be determined.

In addition, robotic system 20 comprises a controller 26 and at least one computer processor 28 (controller 26 may be part of computer processor 28), via which components of the robotic and ultrasound imaging systems and a user (e.g., a healthcare professional) operatively interact with each other. Typically, controller 26 is able to control and activate motor system 27. In turn, controller 26 reads the location and orientation (e.g., via rotary encoder data) of each one of motors 21, 23 and 25 that drive the ultrasound probe and outputs the information to a computer processor 28. Additionally, computer processor 28 receives scanned data from ultrasound imaging system 30. Based on the location information of the ultrasound probe and the scanned data, the computer processor performs data analysis and processing of the images to create three-dimensional high-resolution images for identifying endometriosis lesions within the images. For some applications, ultrasound robotic system 20 comprises a streamer configured to receive the scanned data from ultrasound imaging system 30 and stream the data to the computer processor (e.g., in real time). For some applications, robotic imaging system 20 comprises a user interface via which a user interacts with components of robotic system 20 (e.g., the user interface comprises a display 32, shown in FIG. 2).

Reference is still made to FIG. 1A. As described hereinabove, in accordance with some applications of the present invention, ultrasound contrast agents are used to enhance endometriosis lesions within the images produced by robotic ultrasound system 20. Typically, administering of the ultrasound contrast agents is carried out between acquiring two sets of images, for processing the two sets of images to produce a contrast-enhanced image of the endometriosis lesions.

Typically, prior to the contrast agent having been administered to the subject, computer processor 28 (or controller 26, which is typically a component of computer processor 28) drives robotic system 20 to acquire a first set of ultrasound images of the subject's pelvis and/or abdomen while moving along a first predefined path relative to the pelvis and/or abdomen of the subject, such that the location and orientation of the ultrasound probe relative to the subject's pelvis and/or abdomen at the acquisition of each of the ultrasound images belonging to the first set of ultrasound images is known.

Subsequent to acquiring the first set of ultrasound images, contrast agents are administered to the subject. Subsequent to the contrast agent having been administered to the subject, computer processor 28 drives the robotic system to acquire a second set of ultrasound images of the subject's pelvis and/or abdomen while moving along a second predefined path relative to the subject's pelvis and/or abdomen, such that the location and orientation of the ultrasound probe relative to the subject's pelvis and/or abdomen at the acquisition of each of the ultrasound images belonging to the second set of ultrasound images is known. Typically, the first and second predefined paths are the same, such that the post contrast agent scanning is performed at the same location and orientation as the pre contrast agent scanning. Additionally, robotic system 20 maintains the ultrasound probe at a constant orientation in space as the robotic system moves the ultrasound probe along the same first and second predefined paths.

Computer processor 28 is configured to process the data of the first and second sets of images to produce contrast-enhanced images of the endometriosis lesions. For some applications, computer processor 28 is configured to process the data to subtract images that were acquired from each location and orientation relative to the subject's pelvis and/or abdomen within the first set of ultrasound images from the images that were acquired from the same location and orientation relative to the subject's pelvis and/or abdomen within the second set of ultrasound images, such as to generate a set of subtraction images, each of the subtraction images corresponding to a given location and orientation of the ultrasound probe relative to the subject's pelvis and/or abdomen, and to identify endometriosis lesions within the subtraction images. Typically, by having the post contrast agent scanning performed at the same exact location and orientation as the pre contrast agent scanning, small lesions (e.g., lesions having a diameter of 1 mm), are identified within the subtraction images.

For some applications, the computer processor identifies endometriosis lesions by analyzing the first and second sets of ultrasound images but without generating subtraction images from the first and second sets of ultrasound images. Additionally, or alternatively, computer processor 28 is configured to process the data by using an Artificial Intelligence (AI) algorithm, or by additional processing techniques as will be described hereinbelow.

Reference is now made to FIG. 1B, which is a flowchart showing steps of imaging and diagnosis of endometriosis lesions that are performed using the robotic ultrasound system, in accordance with some applications of the present invention.

For some applications, in step 100 (typically following setup of the robotic system, software upload and ultrasound image calibration), initial pre-contrast agent injection scanning is performed by robotically moving the ultrasound transducer over a region of interest (e.g., the pelvis and abdomen of the subject). In step 102 the acquired scanned data is streamed to a computer processor of the robotic-assisted ultrasound system. Subsequently, in step 104, ultrasound contrast agents are injected (e.g., intravenously, locally, and/or in an abdominal/intra-uterine manner) and are allowed to accumulate in the target tissue of the endometriosis tissue. In step 106 post-contrast agent injection scanning is performed by robotically moving the ultrasound transducer over the region of interest. For some applications, ultrasound scanning is performed at a pulse inversion scanning mode for better contrast enhancement. The scanned data is streamed to the computer processor (step 108). In step 110 data analysis is performed by the computer processor resulting in identification and mapping of the lesions.

In accordance with some applications of the present invention, the data analysis step 110 can include the following steps:
1) Data analysis by converting video format data from the ultrasound scanning to 3D DICOM images based on robotic arm location and assembling images from the same plan (typically, for obtaining images with a resolution of 10 microns which is generally up to 100 times higher than the resolution of standard ultrasound imaging).
2) Subtraction of images pre- and post-contrast enhancement by the contrast agents, to emphasize lesions echogenicity and remove background tissue.
3) Automatic segmentation of internal organs for identification of lesions within the imaged organs, and automatic segmentation of the lesions according to their echogenicity. Display to a user (e.g., operator of the system), and training an AI for deep learning to identify the endometriosis lesions (or manual delineation of the lesion on the images).
4) Analysis tools such as distance measurements and volumes.

For some applications, performing of the scanning and/or processing and analysis of the scanned data includes any one of, or a combination of, the following procedures:
Scanning multiple times and selecting the data with least artifacts, to be combined to one volume of data.
In the case of identifying artifacts or poor image quality in real time, using automated control (e.g., artificial-intelligence driven control) to overcome such issues, e.g. by rotating the ultrasound probe or changing the speed of scanning (e.g., moving the probe more slowly).
Motion and/or deformation correction. Typically, accounting for motion (e.g., by implementing motion correction algorithms) is enabled due to high redundancy and overlap of data, and additionally due to the high resolution of the images. Additionally, or alternatively, in order to reduce motion that would require correcting, the robotic systems described herein are coupled (e.g., fixed) to the subject being examined (rather than to a bed or examination chair on which the subject is positioned).
Synchronization of the scanning with the heart rate and breathing rate of the subject.
Initially, fast acquisition of image data, followed by identification by artificial-intelligence algorithms of area suspected to be endometriosis lesion, followed by slowing the acquisition of image data and taking steps to acquire data of better quality and increased quantity (e.g., by slowing scanning and/or changing the orientation of the ultrasound probe).
Scanning in several frequencies and combining the data into a single volume. For example, scanning at a relatively high frequency results in a higher image resolution but imagining at less depth of the scanned area, and vice versa. For some applications of the present invention, artificial-intelligence (AI) algorithms are used to train the system to produce high resolution images from lower resolution images.
Segmentation of internal organs and using artificial-intelligence algorithms to search and identify endometriosis lesions within the segmented images.
Performing the ultrasound scanning at a pulse inversion scanning mode for better contrast enhancement.
Performing the ultrasound scanning by moving the ultrasound probe at a velocity of 0.25-10 mm/sec.
Using a transmitting ultrasound probe and a receiving ultrasound probe. For example, one of the probes may be an intraluminal probe (e.g., a vaginal probe or a rectal probe) and the other an abdominal probe. Or a probe positioned on the lower back of a subject while the second probe is placed on the abdomen.
Using an external abdominal ultrasound probe and an intraluminal ultrasound probe (e.g., a vaginal ultrasound probe), and combining data from both ultrasound probes to one volumetric data.

### Treatment and Intraoperative Real-Time Detection

In accordance with some applications of the present invention, the robotic imaging ultrasound system provided in accordance with some applications of the present invention, is also configured for facilitating treatment of the detected endometriosis lesions, e.g., by applying treatment energy for treatment of the detected endometriosis lesions.

For some applications, the same ultrasound probe that is used for imaging is also used for applying ultrasound treatment energy by changing parameters of the applied ultrasound energy. Alternatively, a separate ultrasound probe is provided for applying ultrasound treatment energy effective for treating the endometriosis lesions. For example, in either case, the ultrasound probe is configured to apply targeted focused ultrasound (e.g., HIFU or LIFU ultrasound), to ablate the endometriosis lesions.

Additionally, or alternatively, the robotic systems described herein are configured to robotically aim a laser source for ablation of the lesion (in open surgery), and/or or a needle for a drug injection directly into the endometriosis lesions.

Further additionally or alternatively, the ultrasound contrast agents described herein as being used to enhance imaging of the endometriosis lesions within acquired ultrasound images, are also used for delivering targeted treatment to the lesions, thus achieving a selective, non-thermal, mechanical or pharmaceutical therapeutic effect. For example, ultrasound contrast agents (e.g., the targeted contrast agents) can deliver various bioactive substances, thereby providing cell-specific drug delivery.

Drugs delivered to the endometriosis lesions, e.g., using the ultrasound contrast agents, may be released at the site of the endometriosis lesion in at least two modes of operation: one is based on improved drug permeability, and the second is based on ultrasound-activated drug at the target site (e.g., sonodynamic therapy).

It is noted that in accordance with some applications of the present invention, treatment planning is based on the imaging performed in accordance with techniques, apparatus and systems described herein. Additionally, ultrasound imaging is used to monitor the application of treatment and the results of the applied treatment.

In accordance with some applications of the present invention, the robotic imaging ultrasound system provided in accordance with some applications of the present invention, is also configured for facilitating treatment of the detected endometriosis lesions, e.g., by applying treatment energy for treatment of the detected endometriosis lesions.

For some applications, selective treatment targeting of the endometriosis lesions is enhanced by motion detection of an organ or the subject (typically by using artificial-intelligence algorithms), and robotically adjusting the ultrasound probe to directly target the lesion.

In summary, in accordance with some applications of the present invention, the following sequence of steps may be performed in the course of treatment of endometriosis lesions:
Preparation of a treatment plan based on verification and correction of endometriosis lesions identified through artificial intelligence or manually marked.
Treatment execution (e.g., by the delivery of focused ultrasound based on the treatment plan to ablate the lesion and/or activation of contrast agents e.g., by cavitation, sonodynamic therapy (described in further detail hereinbelow), and/or by local drug injection, and/or application of laser energy).
Treatment monitoring by ultrasound imaging throughout the course of treatment.

For some applications, the robotic imaging ultrasound system provided in accordance with some applications of the present invention, is also configured for concomitant intraoperative real-time detection of the endometriosis lesions during surgical procedures such as laparoscopic surgical procedures. In such a manner, it is ensured that the therapeutic process indicated in a preoperative surgical plan is performed exclusively on lesion tissue, and the surgical procedure is not relying solely on supposed positioning of the lesions as indicated by preoperative images. This is particularly important in cases of abdominal surgeries, since for some such applications, preoperative images may not be completely accurate due to movements during the procedure. (Specifically, endometriosis lesions are very difficult to locate and identify due to abdominal adhesions.) In such a manner, damage to healthy tissue is generally avoided. Intraoperative real-time detection during surgical treatment of the lesion is thus highly advantageous in such procedures.

In summary, in accordance with some applications of the present invention, the following sequence of steps may be performed in the course of intraoperative real-time detection of endometriosis lesions during a treatment procedure (such as laparoscopic surgery):
Robotic-assisted or standard manual ultrasound imaging in order to identify lesion area in general with and without targeted ultrasound contrast agents.
Administering (e.g., intravenously or locally or abdominally) targeted (or non-targeted) ultrasound contrast agents.
Ultrasound imaging with an ultrasound scanner (either robotic-assisted or a standard manual scanner) after administration of the ultrasound contrast agents.
Employing pulse inversion scanning mode for better contrast enhancement.
Applying treatment to the lesion by applying focused ultrasound according to a treatment plan, and additionally for activation of contrast agents either by cavitation, sonodynamic therapy or other. (Other treatment options such as laser or drug injection, or lesion resection may be applied).
On-going treatment monitoring by ultrasound imaging throughout the treatment may be performed.

### Contrast Agents

As described hereinabove, ultrasound contrast agents are used for enhanced identification of the lesions in the images that are acquired and processed, in accordance with applications of the present invention. The contrast agents are used in combination with a robotic ultrasound system described herein, (e.g., any of the robotic systems described in FIGS. 1A-9B). The combination of using ultrasound contrast agents that accumulate in the endometriosis lesions together with scanning the lesions and processing the scans with the robotic ultrasound system provided herein, produces super resolution (e.g., a resolution of 10 microns) three-dimensional images of the lesions.

The contrast agents are typically administered, e.g., by injection, to the patient subsequently to acquiring initial ultrasound scans of the patient. Following administering of the contrast agents, additional ultrasound scans are acquired and the data is processed as described hereinabove.

For some applications, the ultrasound contrast agents comprise targeted ultrasound contrast agents that are targeted to the lesions by having antibodies (or other target-specific molecules) conjugated to the contrast agents. The antibodies can be targeted to various targets in relevant tissue associated with endometriosis. For example, the antibodies may be targeted to ectopic endometrium cells, fibrotic tissue, inflammation, angiogenesis, neurogenesis, stromal cells, or a combination of such targets (or any other molecular target that differentiates the lesion from its surrounding tissue).

For some applications, the contrast agents include a drug for release at the target cell or activated at the target (e.g., sonodynamic therapy, e.g. 5-ALA).

For some applications, the contrast agents are multi-modal and suitable for use in various imaging modalities including ultrasound, CT, X-ray, and/or MRI.

For some applications, the ultrasound contrast agents comprise free (non-targeted) microbubbles, that act as echo-enhancers. For some applications, the ultrasound contrast agents comprise targeted microbubbles. For example, VEGFR2 targeted microbubbles are used in accordance with some applications of the present invention. Endometriosis lesions have been shown to have extensive angiogenesis which was shown to be associated with high expression of Vascular endothelial growth factor (VEGF) and its receptors in the lesion's blood vessels. Thus, it is hypothesized by the inventors that VEGFR2 targeted microbubbles (such as BR55 (Bracco Research inc.)) will accumulate within the vasculature of the lesion in a higher concentration and for a longer duration than adjacent tissue due to the extensive angiogenesis in endometriosis lesions. Accumulation of the VEGFR2 targeted microbubbles within the endometriosis lesions will in turn result in locally enhanced ultrasound images.

For some applications, microbubbles, or nanobubbles are used to specifically target endometriosis cells. For some such applications, targeted nano-bubbles are attached to a ligand that specifically targets receptors on the endometriosis cells. The nanobubbles pass through endothelial gaps of blood vessels to reach the lesion cells. The ligand that is coupled to the nanobubbles binds the endometriosis cells receptors. It is hypothesized by the inventors that providing nanobubbles that are conjugated to a ligand that can selectively bind estrogen receptor (ER)β in endometriosis cells and/or IL-1R in endometriosis cells will result in effective ultrasound enhancement (as estrogen receptor (ER)β and IL-1R have been shown to have an increased expression on endometriosis cells compared to healthy cells).

As described hereinabove, for some applications, during the treatment stage, the ultrasound probe is configured to apply targeted focused ultrasound (e.g., LIFU ultrasound) toward the identified lesions. For some applications, the targeted focused ultrasound causes cavitation of tissue in the vicinity of any microbubbles or nanobubbles that the ultrasound waves impact. Since microbubbles or nanobubbles preferentially target endometriosis cells, this preferentially destroys the endometriosis cells. Thus, there is a two-fold targeting of the endometriosis cells—first the endometriosis cells are imaged and the ultrasound is preferentially directed toward the endometriosis cells, and secondly, the endometriosis cells are preferentially targeted when causing the cavitation of tissue and the destructions of cells. In such a manner, for some applications, contrast agents are used in both detection and ultrasound-mediated therapy of endometriosis lesions. The lesions that are identified in the imaging procedure are aimed at by the robotic systems provided herein to apply the ultrasound treatment energy to cause contrast-agent mediated destruction of the tissue. (e.g., LIFU in the case of using the microbubbles as described herein) to treat the lesions.

It is noted that for some applications, the microbubbles described herein may be administered intravenously and/or via an intravaginal approach. It is further noted, that for some applications, use of contrast-enhancing microbubbles (or other targeted or non-targeted contrast agents) are used to train an artificial intelligence and/or machine-learning algorithm to identify endometriosis lesions, such that actual use of the contrast agents is only required during the training stage, and avoided once the system is trained.

### Robotic Systems

Reference is now made to FIGS. 2-9B, which are schematic illustrations of various apparatuses comprising robotic systems configured for use with one or more ultrasound probes for identifying endometriosis lesions within the body of a subject, in accordance with some applications of the present invention. Robotic systems provided herein typically control scanning of the subject with the ultrasound probes, such that the location and orientation of the ultrasound probe relative to the subject at the at the acquisition of the ultrasound images is known.

Additionally, for some applications, the robotic systems shown in FIGS. 2-9B, are used in combination with contrast enhanced ultrasound imaging as described herein. Typically, the ultrasound images that are acquired using the robotic system of FIGS. 2-9B, are processed and analyzed as described herein (e.g., in accordance with techniques described with reference to FIG. 1B). In such a manner, the endometriosis lesions are identified within the images produced by the robotic systems shown in FIGS. 2-9B.

For some applications, as described herein, the robotic systems are configured to identify the endometriosis lesions based on pre-training of the system to identify endometriosis lesions based only on ultrasound images that are acquired in the absence of contrast agents (such that use of contrast agents is not required). Reference is first made to FIG. 2, which is a schematic illustration of an apparatus comprising a robotic system 120 configured for identifying endometriosis lesions within the body of a subject, in accordance with some applications of the present invention. Robotic system 120 is configured for use with one or more ultrasound probes 22. Robotic system 120 typically comprises a tray 50 that defines internal channels and is configured to be placed on a pelvis and/or an abdomen of the subject. Additionally, robotic system 120 typically comprises an ultrasound probe supporting portion 36 that is configured to hold ultrasound probe 22, to move along the internal channels of tray 50. Typically, ultrasound system 120 comprises a control console 29 that includes computer processor 28 and controller 26 (it is noted that controller 26 may be a component of computer processor 28) and display 32.

In accordance with some applications of the present invention, tray 50 is placed on pelvis and/or abdomen 14 of a female subject 10, while subject 10 is laying on a gynecological chair 12, and an ultrasound probe supporting portion 36 holds ultrasound probe 22 and moves probe 22 along the internal channels defined by the tray. Computer processor 28 is configured to drive the robotic system to acquire ultrasound images of the subject's pelvis and/or abdomen while probe 22 moves along the internal channels defined by the tray, such that the location and orientation of ultrasound probe 22 at the acquisition of each of the ultrasound images relative to the subject's pelvis and/or abdomen is known. Computer processor 28 is configured to process the ultrasound images as described hereinabove to create images of high resolution (e.g., resolution of 10-micron), in which the endometriosis lesions are identified. Typically, tray 50 is configured to maintain the ultrasound probe at a constant orientation in space as the robotic system moves the ultrasound probe. For some applications, tray 50 is configured to maintain the orientation of the ultrasound probe (i.e., the transducer of the ultrasound probe) such that it is substantially parallel to a tangent to a center of a pelvis and/or an abdomen of the subject as the robotic system moves the ultrasound probe.

As shown in blow-up A of tray 50, for some applications, tray 50 is shaped to conform to the curvatures of a pelvis and/or abdomen 14 such that when placed on a pelvis/abdominal area on subject 10, tray 50 is coupled to the skin of the subject generally without gaps between tray 50 and the subject. A bottom surface of tray 50 typically comprises an ultrasound transparent material such as nylon or mesh and acoustic coupling gel is typically applied to both sides of the transparent material (i.e., on the side that is placed in contact with the subject and on the side over which ultrasound probe 22 is moved). Blow up A shows parallel movement of ultrasound probe 22 along a predefined path provided by tray 50 as indicated by arrow A1. As the robotic system moves the ultrasound probe along the tray, the robotic system typically maintains ultrasound probe 22 at a constant orientation in space. For example, ultrasound probe 22 is maintained at a constant orientation such that it is substantially parallel to a tangent to a center of a pelvis and/or an abdomen of the subject as the robotic system moves ultrasound probe 22, as indicated in blow up A of FIG. 2.

For other applications, as shown in blow-up B, tray 50A, is similar to tray 50, except that the base of tray 50A is flat and is not curved to conform to the shape of the pelvis and/or abdomen. For some such applications, a water bath 38 is placed underneath tray 50A (in order to close any gaps between the tray and the abdomen and provide acoustic coupling) and flat base of tray 50A is configured to maintain the ultrasound probe at a constant orientation in space as the robotic system moves the ultrasound probe. Typically, the flat base of tray 50A is configured to maintain ultrasound probe 22 substantially parallel to the tangent to the center of the subject's pelvis and/or abdomen as the robotic system moves the ultrasound probe along a predefined path provided by tray 50A, and indicated by arrow A2 in blow-up B.

For some applications, maintaining the ultrasound probe at a constant orientation in space over the course of the procedure facilitates the generation of high-resolution three-dimensional images. For example, this typically facilitates the combination of images acquired from respective positions with each other such as to generate a three-dimensional image, because the orientations of the images in space with respect to each other are fixed. For some applications, maintaining the transducer of the ultrasound probe substantially parallel to the tangent to the center of the subject's pelvis and/or abdomen as the robotic system moves the ultrasound probe facilitates the generation of high-resolution three-dimensional images. For example, this typically facilitates acquiring a large amount of useful and high-resolution imaging data within each image, because the ultrasound transducer is substantially directly facing the coronal plane of the subject's abdomen and/or pelvis (or other scanned body area), since the tangent to the center of the subject's pelvis and/or abdomen (or other scanned body area) is typically parallel to the coronal plane of the subject's body at the abdomen and/or pelvis (or other scanned body area).

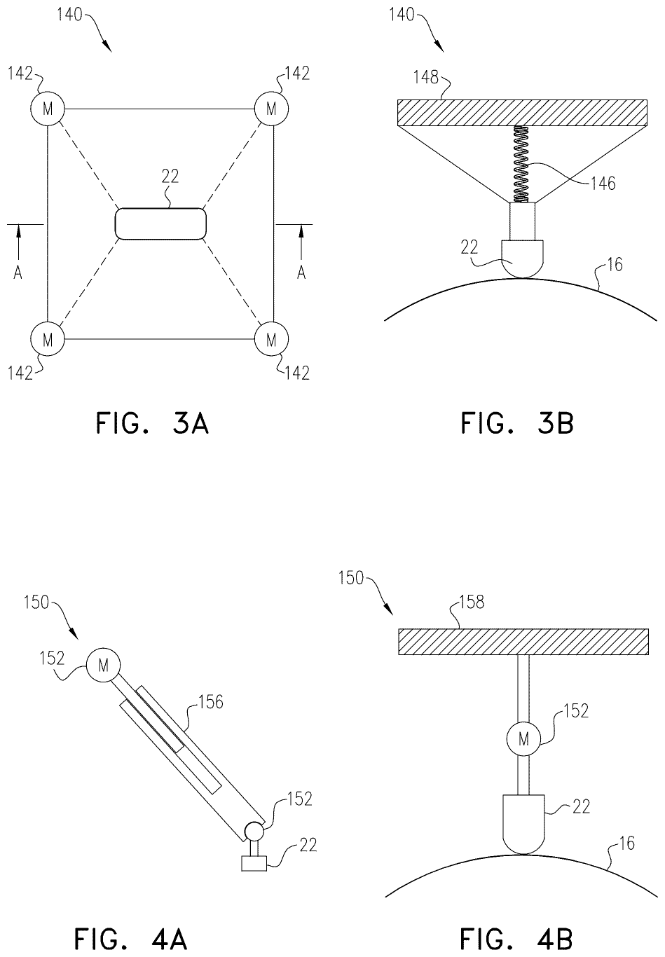

Reference is now made to FIGS. 3A-3B, which are schematic illustrations of components of a robotic system 140 configured for use for identifying endometriosis lesions within a body of a subject, in accordance with some applications of the present invention. FIG. 3A shows a top view of components of robotic system 140, and FIG. 3B shows a side view of components of robotic system 140. As shown, components of robotic system 140 include four motors 142, a tray 148, and a spring 146. Robotic system 140 is configured for use with ultrasound probe 22 for scanning over skin 16. As shown, robotic system 140 is configured for movement along the X axis and Y axis in one plane, by having four motors 142, one at each corner of the plane connected to ultrasound probe 22 by cables 143. The Z axis is passive, having only an encoder (e.g., a wire encoder). Spring 146 typically facilitates movement in an upward direction by balancing the transducer weight while being pushed against the surface of skin 16.

Reference is now made to FIGS. 4A-4B, which are schematic illustrations of components of a robotic system 150 configured for use for identifying endometriosis lesions within a body of a subject, in accordance with some applications of the present invention. FIG. 4A shows a top view of components of robotic system 150, and FIG. 4B shows a side view of components of robotic system 150. As shown, components of robotic system 150 comprise a platform 158 and a robotic arm 156 for controlling movement of ultrasound probe 22 across skin 16, using rotation motors 152. Robotic system 150 has a motorized Z axis with distance sensor and an encoder. Arm rotation motor 152 on the base of arm 156 facilitates motion of ultrasound probe 22, while allowing elongation of arm 156 in Z and XY axes.

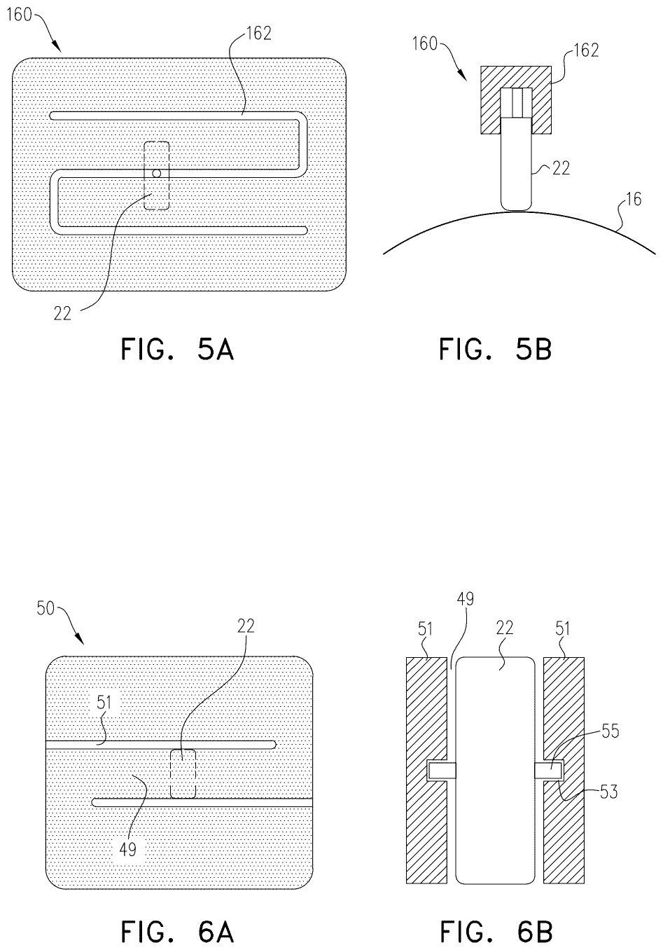

Reference is now made to FIGS. 5A-5B, which are schematic illustrations of components of a robotic system 160 configured for use for identifying endometriosis lesions within a body of a subject, in accordance with some applications of the present invention. FIG. 5A shows a top view of components of robotic system 160, and FIG. 5B shows a side view of components of robotic system 160. Robotic system 160 comprises trail 162, similar to a monorail, along which ultrasound probe 22 is moved. Trail 162 is placed above skin 16 and the ultrasound transducer is moved along the predefined path created by trail 162 such that the location and orientation of the ultrasound probe at the acquisition of each of the ultrasound images relative to the subject is known. Typically, trail 162 can be tailored to specifically fit the size and shape of a subject and have a predetermined path to specifically match the subject's needs.

Reference is now made to FIGS. 6A-6B, which are schematic illustrations of tray 50 (typically, flat tray 50A) described herein with reference to FIG. 2, in accordance with some applications of the present invention. FIG. 6A shows a top view of tray 50, and FIG. 6B shows a side view of tray 50. Tray 50 has internal channels 49 which define a determined path along which ultrasound probe 22 moves. For some applications, the channels are separated from each other by walls 51. The walls define grooves 53 that are configured to receive protrusions 55 from the ultrasound probe. The ultrasound probe moved along the channels in a controlled manner by protrusions 55 sliding along grooves 53. As described herein above, tray 50 ensures that the location and orientation of the ultrasound probe at the acquisition of each of the ultrasound images relative to the subject is known.

Tray 50 (and trail 162) can be manufactured to be tailored to match the body shape of the subject, e.g., according to three-dimensional vision-based mapping that is carried out before the ultrasound scanning using the robotic system described herein.

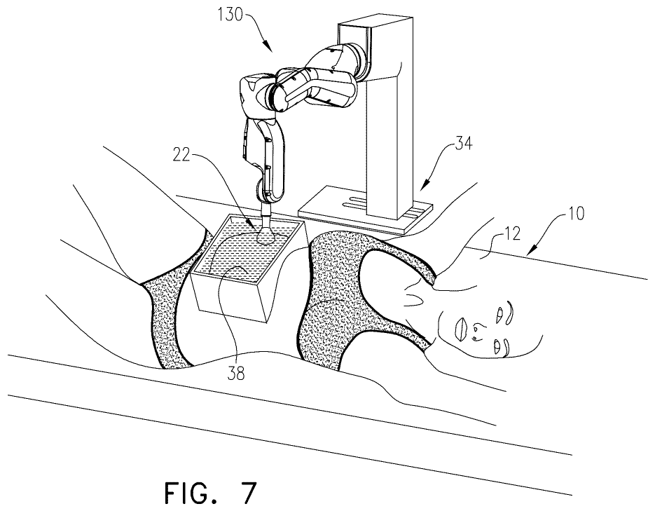

Reference is now made to FIG. 7, which is a schematic illustration of an apparatus for identifying endometriosis lesions within a body of a subject, comprising a robotic system 130 configured for use with for one or more ultrasound probes 22, in accordance with some applications of the present invention. As shown, robotic system 130 is configured for use with a water-filled compartment, e.g., water bath 38. As described hereinabove with reference to FIG. 2, for some applications, in a flat configuration thereof, tray 50A is used with a water bath to ensure coupling of the ultrasound probe to the skin of subject 10 at the region being scanned (e.g., the abdomen/pelvis area). It is noted that, as shown in FIG. 7, use of water bath 38 is not limited to use with a tray but may also be used with a robotic system having a robotic arm fixed to gynecological chair 12 (as shown in FIG. 7) being configured for motion in XY directions within the water bath.

As shown in FIG. 7, the base of the robotic arm is coupled to a base 34, e.g., via a trail upon which the base of the robotic arm moves, as shown. For some applications, the ultrasound probe is maintained in a fixed orientation relative to base 34 (e.g., using a robotic arm that has a double-parallelogram structure as described hereinbelow). Thus, the ultrasound probe is typically maintained in a fixed orientation with respect to the back of the gynecological chair and therefore maintained in a substantially fixed orientation in space over the course of the procedure. For some applications, maintaining the ultrasound probe at a constant orientation in space over the course of the procedure facilitates the generation of high-resolution three-dimensional images. For example, this typically facilitates the combination of images acquired from respective positions with each other such as to generate a three-dimensional image, because the orientations of the images in space with respect to each other are fixed.

For some applications, the transducer of the ultrasound probe is maintained substantially parallel to a tangent to a center of an abdomen of the subject as the robotic system moves the ultrasound probe. For example, the transducer of the ultrasound probe may be maintained substantially parallel to the tangent to the center of the subject's pelvis and/or abdomen (or other scanned body area) by fixing the orientation of the ultrasound probe relative to base 34 (which is fixed to the gynecological chair), and by assuming minimal movement between the subject's pelvis and/or abdomen and the back of the gynecological chair over the course of the procedure. For some alternative applications, base 34 is coupled directly to the subject, such that the base of the robotic arm (and thereby the ultrasound transducer) is maintained in a substantially fixed orientation with respect to the subject's abdomen over the course of the procedure.

For some applications, maintaining the transducer of the ultrasound probe substantially parallel to the tangent to the center of the subject's pelvis and/or abdomen (or other scanned body area) as the robotic system moves the ultrasound probe facilitates the generation of high-resolution three-dimensional images. For example, this typically facilitates acquiring a large amount of useful and high-resolution imaging data within each image, because the ultrasound transducer is substantially directly facing the coronal plane of the subject's abdomen and/or pelvis the coronal plane of the subject's body at the abdomen and/or pelvis (or other scanned body area). As described hereinabove, for some applications, tray 50A (shown in FIG. 2) is used to maintain the ultrasound probe (i.e., the transducer of the ultrasound probe) in a fixed orientation in space (e.g., substantially parallel to a tangent to a center of an abdomen of the subject) over the course of the procedure.

For some applications, water bath 38 is filled with water and is structured such that an upper side of bath 38 (i.e., a side through which ultrasound probe 22 is inserted into the bath) is open, in order to allow ultrasound probe 22 to be inserted into and move within bath 38. A bottom surface of bath 38 (i.e., the side that comes in contact with the subject) is typically made of a flexible ultrasound transparent material. The bottom side of bath 38 is typically coupled to the skin with acoustic coupling gel that is applied between the skin and the water bath.

FIG. 7 shows subject 10 in a supine position with the water bath positioned on an abdomen of the subject, in accordance with some applications of the present invention. For other applications, subject 10 is positioned in a prone position with the water bath positioned between the bed and subject 10 and the transducer moves below subject 10 to scan images.

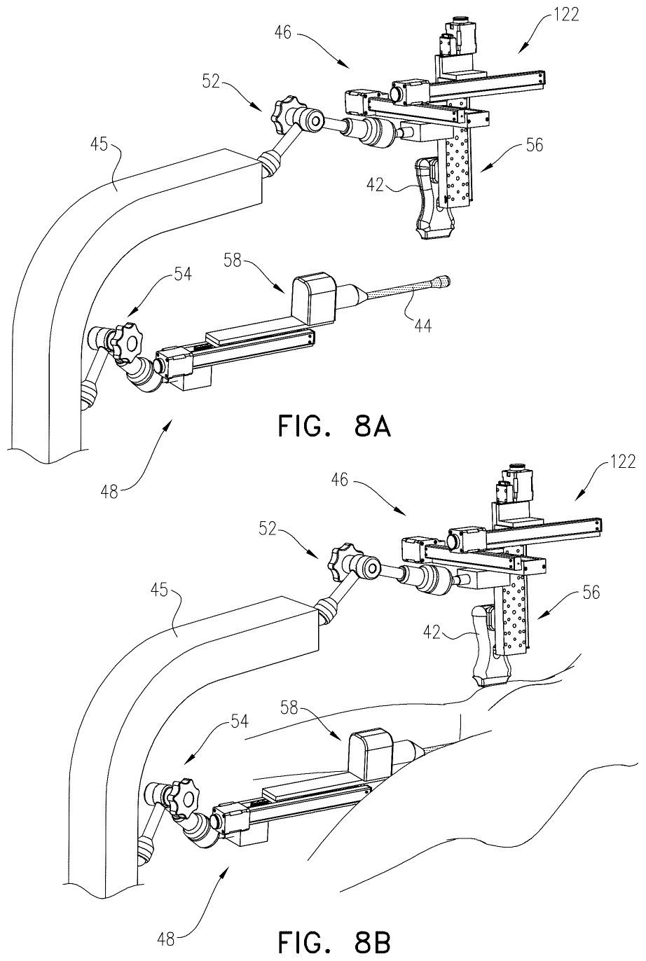

Reference is now made to FIGS. 8A and 8B. FIG. 8A is a schematic illustration of an apparatus for identifying endometriosis lesions within a body of a subject comprising a robotic system 122 configured for use with first and second ultrasound probes (e.g., an external abdominal ultrasound probe 42 and an intraluminal ultrasound probe 44). FIG. 8B is a schematic illustration of robotic system 122 being used to scan the subject, showing first probe 42 above the pelvis and/or abdomen of the subject and second probe 44 inserted into a lumen of the subject (e.g., rectum or vagina), in accordance with applications of the present invention.

Robotic system 122 comprises an abdominal portion 46 comprising one or more abdominal robotic arms 52 and an abdominal ultrasound probe supporting portion 56 that is configured to hold first ultrasound probe 42 above an abdomen of the subject. Robotic system 122 additionally comprises an intraluminal portion 48 comprising one or more intraluminal robotic arms 54 and an intraluminal ultrasound probe supporting portion 58, the intraluminal portion being configured to insert second ultrasound probe 44 into a lumen of the subject selected from the group consisting of: a rectum and a vagina.

Robotic system 122 additionally comprises at least one computer processor 28 (computer processor 28 is not shown in FIGS. 8A-B) configured to determine the positions and orientations of the abdominal and intraluminal ultrasound probes 42 and 44, with respect to each other, and to drive first ultrasound probe 42 to acquire abdominally-acquired ultrasound images of the subject's pelvis and/or abdomen while driving abdominal portion 46 of robotic system 122 to move first ultrasound probe 42 relative to the subject's pelvis and/or abdomen. The computer processor is additionally configured to drive second ultrasound probe 44 to acquire intraluminally-acquired ultrasound images of the subject's pelvis and/or abdomen while driving intraluminal portion 48 of robotic system 122 to move second ultrasound probe 44 relative to the subject's pelvis and/or abdomen. The computer processor is then configured to generate three-dimensional ultrasonic imaging data based on a combination of the abdominally-acquired ultrasound images and the intraluminally-acquired ultrasound images (typically using processing techniques described herein) to identify endometriosis lesions based upon the three-dimensional ultrasonic imaging data. Typically, ultrasound probe 42 is mounted on abdominal ultrasound probe supporting portion 56 which has a motorized XYZ movement and/or motorized rotation movement. Further typically, ultrasound probe 44 is mounted on abdominal ultrasound probe supporting portion 58 which has a motorized rotation and Z-movement.

For some applications, the abdominal robotic arms 52 and the intraluminal robotic arms 54 are mounted upon the same base 45 as each other, as shown. For such applications, the computer processor typically determines the positions and orientations of the abdominal and intraluminal ultrasound probes 42 and 44, with respect to each other, by virtue of determining the position and orientation of each of the abdominal and intraluminal ultrasound probes 42 and 44 relative to base 45. For some applications, the robotic system maintains the abdominal and/or intraluminal ultrasound probes 42 and 44 at a fixed orientation in space, for example, using the techniques described herein in order to facilitate determining the position and orientation of each of the abdominal and intraluminal ultrasound probes 42 and 44 relative to each other. For some applications, the computer processor determines the position and orientation of each of the abdominal and intraluminal ultrasound probes 42 and 44 relative to a common coordinate system, for example using electromagnetic sensors that are coupled to the ultrasound probes and/or the robotic arms, and/or using a navigation system.

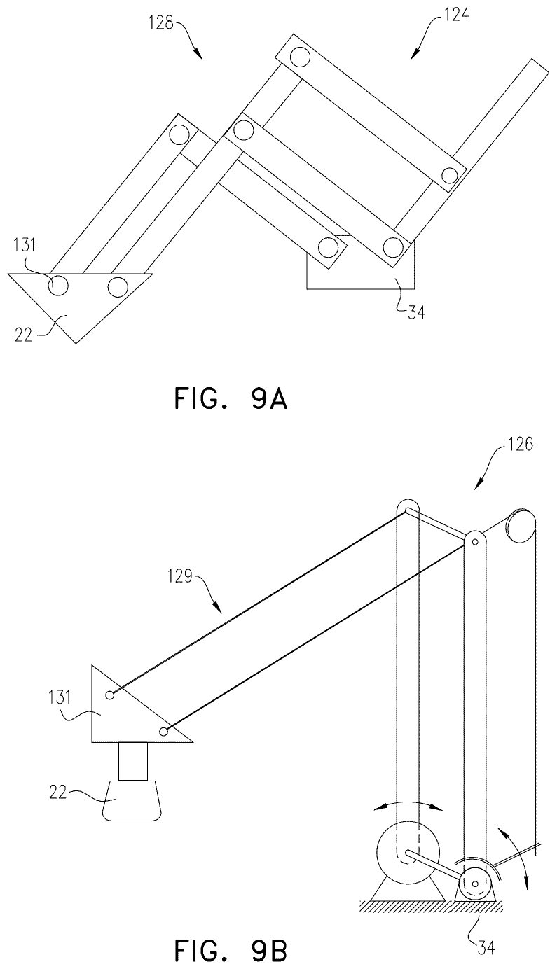

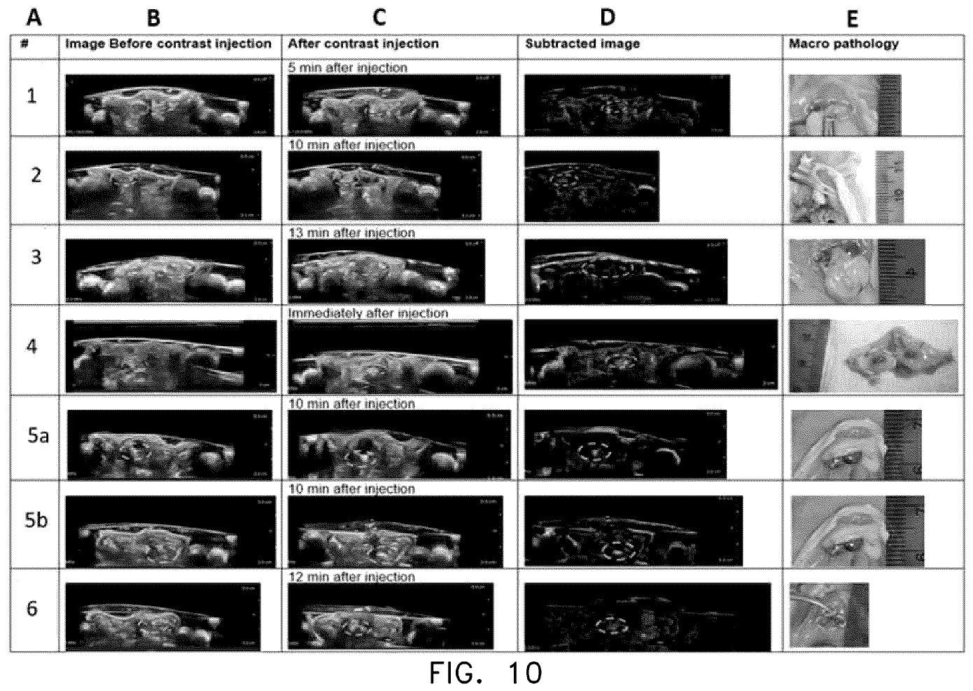

Reference is now made to FIGS. 9A and 9B, which are schematic illustrations of components of an apparatus comprising a robotic system 124 (FIG. 9A) and 126 (FIG. 9B) for identifying endometriosis lesions within a body of a subject, and for use with one or more ultrasound probes (e.g., ultrasound probe 22 shown in FIG. 9B). Robotic system 124 and 126 comprise one or more robotic arms 128 and 129, and an ultrasound probe supporting portion 131 that is configured to hold the ultrasound probe 22. (In FIG. 9A, the ultrasound probe is depicted schematically.) Robotic systems 124 and 126 are configured to maintain an orientation of the ultrasound probe (i.e., the transducer of the ultrasound probe) such that it is substantially parallel to the tangent to the center of the subject's pelvis and/or abdomen (or other scanned body area) as the robotic system moves the ultrasound probe, as described hereinabove with reference to FIG. 7. FIGS. 9A and 9B show robotic system 124 and 126 having robotic arms 128 and 129 having configurations of a double-parallelogram structure for controlling the orientation of the ultrasound probe and maintaining an orientation of the probe (i.e., the transducer of the ultrasound probe) such that it is substantially parallel to the tangent to the center of the subject's pelvis and/or abdomen (or other scanned body area)as the robotic system moves the ultrasound probe. For some applications, the robotic system includes an additional motorized z axis to control the z-axis positioning of the ultrasound probe.

As described hereinabove, typically, base 34 is coupled to the back of gynecological chair 12 (or to a different type of chair or bed upon which the subject is positioned), such that the orientation of the base of the arm is maintained in a fixed orientation with respect to the back of the gynecological chair and therefore maintained in a substantially fixed orientation in space over the course of the procedure. For applications as shown in FIGS. 9A-B, the ultrasound probe is maintained in a fixed orientation relative to base 34 using the double-parallelogram structure. Thus, the ultrasound probe (i.e., the transducer of the ultrasound probe) is maintained in a fixed orientation with respect to the back of the gynecological chair and therefore maintained in a fixed orientation in space over the course of the procedure. As described hereinabove, for some applications, the transducer of the ultrasound probe is maintained substantially parallel to the tangent to the center of the subject's pelvis and/or abdomen (or other scanned body area) as the robotic system moves the ultrasound probe. For example, the transducer of the ultrasound probe may be maintained substantially parallel to the center of the subject's pelvis and/or abdomen (or other scanned body area) by fixing the orientation of the ultrasound probe relative to base 34 (which is fixed to the gynecological chair), and by assuming minimal movement between the subject's pelvis and/or abdomen and the back of the gynecological chair over the course of the procedure. For some alternative applications, base 34 is coupled directly to the subject, such that the base of the robotic arm (and thereby the ultrasound transducer) is maintained in a substantially fixed orientation with respect to the subject's pelvis and/or abdomen over the course of the procedure.

For some applications, maintaining the ultrasound probe at a constant orientation in space over the course of the procedure facilitates the generation of high resolution three-dimensional images. For example, this typically facilitates the combination of images acquired from respective positions with each other such as to generate a three-dimensional image, because the orientations of the images in space with respect to each other are fixed. For some applications, maintaining the transducer of the ultrasound probe substantially parallel to the tangent to the center of the subject's pelvis and/or abdomen (or other scanned body area) as the robotic system moves the ultrasound probe facilitates the generation of high resolution three-dimensional images. For example, this typically facilitates acquiring a large amount of useful and high-resolution imaging data within each image, because the ultrasound transducer is substantially directly facing the coronal plane of the subject's body at the abdomen and/or pelvis (or other scanned body area).

Reference is again made to FIGS. 1A-9B, and the robotic systems described herein. It is noted that in accordance with some applications of the present invention, the robotic systems described herein are attached to the subject being examined (e.g., via base 34, or via tray 50 or 50A), rather than being fixed to an examination bed, a floor, or a console. In such a manner subject movement artifacts are generally avoided because the robotic system moves with the subject's body.

### Coupling of the Ultrasound Probes to the Skin

Reference is again made to FIGS. 1A-9B. As described hereinabove, the robotic systems provided herein are configured for use with at least one ultrasound probe. In accordance with some applications of the present invention, the robotic systems provided herein are configured to be used in ways that ensure optimal coupling between the skin and the ultrasound probes.

In accordance with some applications of the present invention, the ultrasound probe is configured for use while in a hovering position over the skin, with almost no (or only light) contact with the skin. In such a manner, movement and deformation of the skin and underlying body structures undergoing scanning by the probe is generally avoided. For some such applications, a sensor is used with the probe/robotic system for monitoring the distance between the ultrasound probe and the skin. As described hereinabove, for some applications, subtraction images are generated using ultrasound images acquired before and after the administration of contrast agent to the subject. Typically, by avoiding movement and deformation of the skin and underlying body structures undergoing scanning by the probe, changes in the position and/or shape of the skin and underlying body structures between the acquisitions of the pre-contrast and post-contrast images are avoided or minimized, thereby generating accurate subtraction images.

In accordance with some applications of the present invention, optimal coupling between the skin and the ultrasound probe is facilitated by keeping the skin wet (e.g., by using an acoustic coupling gel/cream or by using a water system such as water bath 38 described herein with reference to FIG. 2 and FIG. 7). For some applications, a sensor (e.g., a pressure sensor or an electrical impedance sensor) is included for monitoring the acoustic coupling and the robotic system is configured to discontinue motion of the probe if the interface between the probe and the skin is not sufficiently wet.

In accordance with some applications of the present invention, a uniform wet interface between the ultrasound probe and the skin is maintained by having two acoustic gel dispensers on either side of the ultrasound probe configured to dispense the acoustic gel in the direction in which the probe is moving, in advance of the probe. For some applications, a gel-heating system is configured to warm the gel to increase liquidity of the gel in areas that are contacted by the ultrasound probe.

In accordance with some applications of the present invention, coupling of the ultrasound probe and the skin is facilitated by an ultrasound transparent bag that is filled with acoustic coupling gel and that easily conforms to the shape of a scanned region of the body (e.g., the abdomen). Extra acoustic coupling gel is applied between the bag and the skin and the bag and the ultrasound probe in order to enhance coupling.

Reference is again made to FIGS. 1A-9B. It is noted that techniques and systems described herein are not limited to imaging and treatment of endometriosis lesions. The scope of the present invention includes use of techniques and systems described herein for imaging and treatment of any abnormal tissue and pathogenic cells, including any ectopic tissue or lesions at any location of the body. Additionally, the scope of the present invention includes use of techniques and systems described herein for imaging and treatment of tumors including benign growths and/or malignant growths. Further additionally, the scope of the present invention includes use of techniques and systems described herein for imaging and treatment of inflammations and or thrombi located anywhere within the body.

### Computer Processor

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that algorithms described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described herein, computer processor 28 typically acts as a special purpose ultrasound-imaging computer processor. Typically, the operations described herein that are performed by computer processor 28 transform the physical state of a memory, which is a real physical article, to have a different magnetic polarity, electrical charge, or the like depending on the technology of the memory that is used.

### Experimental Data

Reference is now made to FIG. 10, which is a table showing experimental results of experiments performed by the inventors in accordance with some applications of the present invention and using the apparatus, robotic systems and techniques described herein. The experimental results presented in FIG. 10 demonstrate that the combination of the robotic systems described herein together with the use of contrast agents identify endometrial lesions, including small endometrial lesions that would otherwise remain imperceptible.

A series of protocols are described hereinbelow which may be used separately or in combination, as appropriate, in accordance with applications of the present invention. It is to be appreciated that numerical values are provided by way of illustration and not limitation. Typically, but not necessarily, each value shown is an example selected from a range of values that is within 10% of the value shown. Similarly, although certain steps are described with a high level of specificity, a person of ordinary skill in the art will appreciate that other steps may be performed, mutatis mutandis.

In accordance with some applications of the present invention, the following methods were applied:

Establishing a Rodent Animal Model with Endometriosis.

Immunodeficient SCID-NOD mice were used for establishing the endometriosis model. The surgical procedure was conducted under anesthesia, as follows: a minimal (1 cm) midline incision was made along the Linea Alba. The area surrounding the incision was separated to ensure an adequate detachment of the abdominal wall from the skin. The left uterine horn was exposed, and two micro titanium clips were applied at the utero-tubal junction (just caudal to the fallopian tube) and at the utero-cervical junction (just rostral to the cervix) beneath the extended uterine horn. The segment of the uterine horn situated between the two ligations was excised and placed in a sterile Petri dish containing approximately 100 μL of PBS. A black silk suture with a reverse-cutting needle was used to suture the first implant to an internal anterior abdominal wall.

### Establishing a Setup for Robotic Rodent Scanning Under Anesthesia.

A robotic system for performing experiments on the rodent animal model was established. During the experiments animals were maintained under anesthesia and placed in a temperature-regulated water bath. A Clarius™ linear ultrasound model L15, was used to perform ultrasound imaging. For high-precision control and motion of the ultrasound, a Standa™ linear stage was used employing software: XILAB for precise motion control and monitoring.

Performing Experiments in Rodents with Endometriosis Evaluating Echogenicity of Contrasted Enhanced Lesions:

Experiments were performed on a cohort of six SCID-NOD mice in which the endometriosis model has been established as described hereinabove. A week post-surgery comprehensive ultrasound examination, conducting imaging assessments both before and after intravenous administration of 50-100 microliters of a contrast agent (SonoVue™ contrast agent), was performed.

The results are presented in FIG. 10, which shows distribution of the contrast agent within the ultrasound images of the abdominal cavity, the accumulation of the contrast agent, corresponding to the location of the endometriosis lesion, thus allowing identification of the lesion. The table in FIG. 10 shows results for the six mice (numbered in column A). For each mouse the image before administering the contrast agent is shown (column B), and after administering the contrast agent is shown (column C). As described herein, the image data is processed to form a subtracted image in which the contrast-enhanced lesion is visible, without background noise (column D). Column E shows the actual lesions in the mouse. As shown by the results presented in FIG. 10, the combination of the robotic systems described herein together with the use of contrast agents identify endometrial lesions, including small endometrial lesions that would otherwise may remain imperceptible.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for identifying abnormal tissue within a body of a subject, and for use with at least one ultrasound probe, the apparatus comprising:
a robotic system comprising:
one or more robotic arms; and
an ultrasound probe supporting portion that is configured to hold the ultrasound probe; and
at least one computer processor configured:
to drive the robotic system to acquire ultrasound images of the subject's pelvis and/or abdomen while moving the ultrasound probe relative to the subject's pelvis and/or abdomen, and while maintaining the ultrasound probe in a hovering position over skin of the subject's pelvis and/or abdomen; and
to identify abnormal tissue based upon the ultrasound images.

2. The apparatus according to claim 1, wherein the computer processor is configured to identify endometriosis lesions based upon the ultrasound images.

3. The apparatus according to claim 1, wherein the computer processor is configured to drive the robotic system to acquire ultrasound images of the subject's pelvis and/or abdomen, while maintaining the ultrasound probe in a hovering position over skin of the subject's pelvis and/or abdomen, by driving the robotic system to acquire ultrasound images of the subject's pelvis and/or abdomen, while allowing light contact between the ultrasound probe and the skin of the subject's pelvis and/or abdomen.

4. The apparatus according to claim 1, wherein the computer processor is configured to avoid movement and deformation of the skin and underlying body structures undergoing scanning by the ultrasound probe by maintaining the ultrasound probe in the hovering position over skin of the subject's pelvis and/or abdomen.

5. The apparatus according to claim 1, further comprising a sensor configured to monitor a distance between the ultrasound probe and the skin, wherein the computer processor is configured to maintain the ultrasound probe in the hovering position over skin of the subject's pelvis and/or abdomen based upon the distance between the ultrasound probe and the skin as monitored by the sensor.

6. The apparatus according to claim 1, wherein the ultrasound probe includes a transducer and wherein the robotic system is configured to maintain an orientation of the transducer of the ultrasound probe such that it is substantially parallel to a tangent to a center of the subject's pelvis and/or an abdomen as the robotic system moves the ultrasound probe.

7. The apparatus according to claim 1, wherein the ultrasound probe includes a transducer and wherein the robotic system is configured to maintain an orientation of the transducer of the ultrasound probe such that the ultrasound transducer is substantially directly facing a coronal plane of the subject's body at the subject's abdomen and/or pelvis as the robotic system moves the ultrasound probe.

8. The apparatus according to claim 1, wherein the robotic system is configured to maintain the ultrasound probe at a constant orientation in space as the robotic system moves the ultrasound probe.

9. The apparatus according to claim 1, wherein:
the robotic system comprises a tray configured to be placed on the subject's pelvis and/or abdomen, the tray defining internal channels, and
the ultrasound probe supporting portion is configured to hold the ultrasound probe and to move the ultrasound probe along the internal channels defined by the tray; and
the at least one computer processor is configured to drive the robotic system to acquire the ultrasound images while moving along the internal channels defined by the tray, such that the location and orientation of the ultrasound probe at the acquisition of each of the ultrasound images relative to the subject's pelvis and/or abdomen is known.

10. The apparatus according to claim 9, wherein the tray is configured to be attached to the subject, such that the tray moves with the subject.

11. The apparatus according to claim 9, wherein the tray is shaped to define a curved tray configured to conform to a shape of the pelvis and/or abdomen of the subject, such that when placed on the subject's pelvis and/or abdomen the tray is coupled to skin of the subject generally without gaps between the tray and the subject's skin.

12. The apparatus according to claim 9, wherein a surface of the tray that is placed on the pelvis and/or abdomen of the subject is made of an ultrasound transparent material.

13. The apparatus according to claim 9, wherein the ultrasound probe includes a transducer, and wherein the flat base of the tray is shaped to define a flat base, the flat base configured to maintain an orientation of the transducer of the ultrasound probe such that the transducer is substantially parallel to a tangent to a center of the subject's pelvis and/or abdomen as the robotic system moves the ultrasound probe.

14. The apparatus according to claim 13, further comprising a water-filled compartment configured to be placed between the subject's pelvis and/or abdomen and the flat base of the tray.

15. The apparatus according to claim 1, wherein the apparatus is for use with a contrast agent configured to enhance the abnormal tissue within ultrasound images, and wherein the computer processor is configured:
prior to the contrast agent having been administered to the subject, to drive the robotic system to acquire a first set of ultrasound images of the subject's pelvis and/or abdomen while moving the ultrasound probe;
subsequent to the contrast agent having been administered to the subject, to drive the robotic system to acquire a second set of ultrasound images of the subject's pelvis and/or abdomen while moving the ultrasound probe; and
to identify the abnormal tissue by analyzing the first and second sets of ultrasound images.

16. The apparatus according to claim 15, wherein, by avoiding movement and deformation of the skin and underlying body structures undergoing scanning by the probe, the computer processor is configured to minimize changes in position and shape of the skin and underlying body structures between the acquisitions of the first and second sets of ultrasound images of the subject's pelvis and/or abdomen.

17. The apparatus according to claim 15, wherein the computer processor is configured to subtract images that were acquired within the first set of ultrasound images from the images that were acquired within the second set of ultrasound images, such as to generate a set of subtraction images, each of the subtraction images corresponding to a given location and orientation of the ultrasound probe in space.

18. The apparatus according to claim 17, wherein, by avoiding movement and deformation of the skin and underlying body structures undergoing scanning by the probe, the computer processor is configured to minimize changes in position and shape of the skin and underlying body structures between the acquisitions of the first and second sets of ultrasound images of the subject's pelvis and/or abdomen, to thereby enhance accuracy of the subtraction images.

19. The apparatus according to claim 1, wherein the one or more robotic arms comprise a double-parallelogram structure configured to maintain the ultrasound probe at a constant orientation in space as the robotic system moves the ultrasound probe.

20. The apparatus according to claim 19, wherein the ultrasound probe includes a transducer, wherein the one or more robotic arms comprise a double-parallelogram structure configured to maintain an orientation of the transducer of the ultrasound probe such that it is substantially parallel to a tangent to a center of the subject's pelvis and/or abdomen as the robotic system moves the ultrasound probe.
